(19) United States
(12) Patent Application Publication
(10) Pub. No.: US 2022/0390453 A1
(43) Pub. Date: Dec. 8, 2022

(54) OVARIAN CANCER BIOMARKER AND METHODS OF USING SAME

(71) Applicant: Women's College Hospital, Toronto (CA)

(72) Inventor: Mohammad R. AKBARI, Toronto (CA)

(73) Assignee: Women's College Hospital, Toronto (CA)

(21) Appl. No.: 17/842,373

(22) Filed: Jun. 16, 2022

Related U.S. Application Data
(63) Continuation-in-part of application No. 17/121,012, filed on Dec. 14, 2020.
(60) Provisional application No. 62/949,117, filed on Dec. 17, 2019.

(30) Foreign Application Priority Data
Dec. 17, 2019 (CA) 3065603

(51) Int. Cl. G01N 33/574; G01N 33/68; G01N 33/543

(52) U.S. Cl. CPC G01N 33/57449; G01N 33/6887; G01N 33/54386; G01N 2333/78

(57) ABSTRACT

The present invention provides a novel ovarian cancer marker, Arresten, and related methods, agents, and kits using same. The invention includes methods for detecting or diagnosing ovarian cancer, especially at early stages of the disease. The invention also includes methods of assessing the severity of ovarian cancer and monitoring responses to treatment for ovarian cancer using the biomarker.

### CROSS-REFERENCE TO RELATED APPLICATIONS

This application is a continuation in part of U.S. patent application Ser. No. 17/121,012, filed Dec. 14, 2020, which claims priority from, and the benefit of, U.S. Provisional Patent Application No. 62/949,117, filed Dec. 17, 2019, and Canadian Patent Application No. 3,065,603, filed Dec. 17, 2019. The entire contents of U.S. patent application Ser. No. 17/121,012, U.S. Provisional Patent Application No. 62/949,117, and Canadian Patent Application No. 3,065,603 are hereby incorporated by reference herein.

### FIELD OF INVENTION

This invention relates to a novel ovarian cancer biomarker and to related methods, uses, agents, and kits. More specifically, the invention relates to methods of diagnosing, prognosing, and treating ovarian cancer using the biomarker. The invention also relates to methods of assessing the severity of ovarian cancer and monitoring responses to treatment for ovarian cancer using the biomarker.

### BACKGROUND

Ovarian cancer remains a major health concern worldwide, accounting for 6% of all cancer deaths [1]. It is the second most common gynecological neoplasm, with over 2,800 new cases diagnosed in Canada in 2016 alone, more than 1,800 deaths in the same year [2], and 3,100 new cases diagnosed in Canada in 2020 [34]. In Canada, the 5-year net survival for ovarian cancer is approximately 44%; the survival rate has only modestly increased by 2 to 4% since 1995.

Ovarian cancer is a general term for a group of neoplasms originating from the ovary, the majority (about 90%) of which are classified as epithelial carcinomas. Epithelial ovarian cancers (EOCs) comprise several morphologically distinct groups: serous, mucinous, endometrioid, clear cell, transitional cell, squamous cell, and mixed epithelial neoplasms [3].

High-grade serous ovarian cancer (HGSOC) is an aggressive subtype that accounts for approximately 80% of all ovarian-cancer-related deaths. Worldwide, HGSOC is the eighth most frequent cause of cancer-related deaths in women [4]. Most HGSOC patients present with advanced disease and undergo surgical debulking followed by chemotherapy (e.g., combinations of platinum drugs and paclitaxel) [5]. Their cancers commonly relapse within two years and develop broad chemoresistance, leading to a very poor prognosis [6].

Over the past decade, there has been relatively little improvement in ovarian cancer survival rates. The majority of ovarian cancer cases remain asymptomatic in the early stage and present at an advanced stage, at which point the disease is rarely curable by existing standards of care. As a consequence, ovarian cancer shows the highest mortality rate among gynecologic cancers, with only a 29% 5-year survival rate for advanced ovarian cancer. Importantly, disease outcome is significantly higher (5-year survival rates over 90%) with early diagnosis in stages I and II [7-9].

Accordingly, there exists a need for additional options, including biomarkers, for early detection of primary ovarian cancer. Such biomarkers, including plasma biomarkers, can be useful in periodic screening of asymptomatic women for ovarian cancer and also as diagnostic tools for detecting ovarian cancer in women with a broad range of unspecific symptoms of ovarian cancer.

### SUMMARY

As described in further detail herein, the inventor has surprisingly determined that the Arresten polypeptide or a portion thereof can be used as a biomarker for cancer, particularly ovarian cancer. These discoveries have broad implications in the diagnosis, prognosis, monitoring, and treatment of cancer, including ovarian cancers, and especially type II ovarian cancers, which typically could be diagnosed not earlier than stage II.

Thus, in one aspect, the present application provides a method for determining whether a subject has ovarian cancer, by assaying a diagnostic sample of the subject for Arresten expression, where detection of Arresten expression elevated above normal is diagnostic of cancers, particularly ovarian cancer, in the subject. Also provided is a method of screening a general population for ovarian cancer, by testing a plurality of asymptomatic subjects in accordance with the above method.

Additionally, the present application provides a method for treating ovarian cancer in a subject, by analyzing a diagnostic sample of the subject for Arresten expression, and providing a therapy to the subject when the Arresten expression is elevated above normal. By way of example, the therapy can comprise at least one of: surgical debulking, chemotherapy, and radiation therapy. In certain embodiments, the ovarian cancer is high-grade serous ovarian cancer of stage I-IV (e.g., stage I-IIIb).

The present application also provides a method for assessing the severity of the disease in a patient who has been diagnosed with ovarian cancer. The method includes assaying a biological sample of the patient for Arresten expression prior to treatment and then either: (a) determining a positive or favorable prognosis when Arresten expression in the biological sample is normal, or (b) determining a negative or poor prognosis when Arresten expression in the biological sample is elevated above normal.

The present application further provides a method for assessing the efficacy of therapy to treat ovarian cancer in a subject who has undergone or is undergoing treatment for ovarian cancer. In accordance with this method, the efficacy of the therapy can be assessed by assaying a first diagnostic sample of the subject for Arresten expression after therapy has commenced, obtaining a first level of Arresten expression in the first diagnostic sample, and then comparing the first level with a second level of Arresten expression in a second diagnostic sample of the same subject, where the second diagnostic sample was assayed and the second level was obtained prior to the therapy. A significant decrease of the first detected level, relative to the second level, can indicate that the subject is responding to the therapy to treat ovarian cancer; a minor or no decrease of the first detected level, relative to the second level, can indicate that the subject is not responding to the therapy to treat ovarian cancer. In some embodiments, the first level is below a concentration selected from 80-100 pg/ml, and the second level is above the selected concentration.

The present application also provides a method for assessing the prognosis of a subject who has ovarian cancer, by assaying a diagnostic sample of the subject for Arresten expression. In accordance with this method, the subject's prognosis improves with a decrease in Arresten expression in the diagnostic sample, and the subject's prognosis worsens with an increase in Arresten expression in the diagnostic sample.

Additionally, the present application provides a method for diagnosing ovarian cancer in a human female subject or for screening a human female subject for ovarian cancer when the subject is asymptomatic of ovarian cancer. The method includes: (a) isolating Arresten that is present in a diagnostic sample of the subject by immobilizing the Arresten on a solid surface; (b) forming a complex of the immobilized Arresten with a primary antibody specific for Arresten, where the complex is coupled with an enzyme to form an enzyme complex; (c) incubating the enzyme complex with a substrate for the enzyme; (d) measuring a detectable signal produced by the enzyme acting on the substrate; and (e) calculating a level of Arresten in the diagnostic sample based on the measurement of the detectable signal. In accordance with this method, a level of Arresten elevated above a predetermined cut-off value is diagnostic of ovarian cancer in the subject. The enzyme in the enzyme complex can be conjugated to at least one of the following: (a) the primary antibody; (b) a secondary antibody that binds to the primary antibody; and (c) a protein (e.g., streptavidin or avidin, whether native or modified by glycosylation or deglycosylation) that binds to biotin labelling the primary antibody or the secondary antibody. The immobilized Arresten can be directly immobilized to the solid surface or indirectly immobilized to the solid surface through a capture antibody that is coated on the solid surface and specifically binds to Arresten. In some embodiments, the method further includes comparing the detectable signal to a calibration data set generated using a calibrator (e.g., recombinant human Arresten) at multiple concentrations or levels to assess amount, concentration, or level of Arresten in the diagnostic sample. In some embodiments, the detectable signal is measured by colorimetry, immunofluorescence, bioluminescence, or chemiluminescence.

In a further aspect, the present application provides a method for treating ovarian cancer (e.g., by surgical debulking, chemotherapy, radiation therapy) in a subject who was diagnosed with ovarian cancer by a process that includes: (a) obtaining a serum or plasma sample from the subject; (b) analyzing the sample for Arresten expression; and (c) detecting Arresten expression elevated above normal, thereby diagnosing the subject with ovarian cancer. By way of example, the process can have a sensitivity of at least about 60% and a specificity of at least about 75%. The process can also be based on a receiver operating characteristic (ROC) curve with an area under the curve (AUC) of at least about 0.70.

In the methods of the present invention described herein, the diagnostic sample (e.g., plasma, urine, etc.) can be assayed using an agent reactive with Arresten. In some embodiments, the agent is an antibody or an antigen-binding fragment thereof. The agent can also be labeled with a detectable marker. Furthermore, the diagnostic sample can be assayed using an ELISA, a chemiluminescence assay, an immunohistochemistry assay, and the like. In certain embodiments, Arresten expression is considered to be elevated above normal when the Arresten expression is above a concentration selected from 80-100 pg/ml.

Additionally, in the methods of the present invention described herein, the subject or patient may be at least one of the following: (a) pre-menopausal; (b) asymptomatic of ovarian cancer; (c) not carrying a mutation of BRCA1 or BRCA2 gene; (d) suffering from a non-malignant gynecologic disease, a peritoneal, pleural, or musculoskeletal inflammatory disorder, a pelvic inflammatory disease, a liver, renal, or cardiac disease, or an advanced adenocarcinoma. In some embodiments, the ovarian cancer is a type II ovarian cancer, particularly at stage I, II, Ma, or Mb. By way of example, the ovarian cancer may be undetectable by existing tests, such as CA125 test or a transvaginal ultrasound.

In some embodiments of the present invention, Arresten expression can be detected in conjunction with the detection of at least one additional biomarker, such as CA125 or HE4. In particular, expression of CA125 can be detected in addition to Arresten. An exemplary cut-off value for Arresten expression can be in the range of 80-100 pg/ml; an exemplary cut-off value for CA125 can be in the range of 35-100 U/ml.

In some embodiments of the present invention, the predetermined cut-off value is 80-100 pg/ml; or the predetermined cut-off value is set so that the method has: (a) a sensitivity of at least about 60%; (b) a specificity of at least about 75%; or (c) a sensitivity of at least about 60% and a specificity of at least about 75%. For example, the predetermined cut-off value can be set so that the method has a specificity or sensitivity of about 90% or greater. The predetermined cut-off value can be determined based on a receiver operating characteristic (ROC) curve with an area under the curve (AUC) of at least about 0.70 or at least about 0.85. The predetermined cut-off value can also be based on at least one of the following: (a) a type of ovarian cancer and (b) a stage of ovarian cancer. In some embodiments, the predetermined cut-off value is specific to stage II ovarian cancer and set so that the method has a sensitivity of least about 60%.

In other aspects, the present application provides kits for diagnosing, detecting, and assessing risk of ovarian cancer. For example, in one aspect, the present application provides a kit for use in detecting ovarian cancer, including an agent reactive with Arresten and at least one reagent suitable for detecting expression of Arresten. The kit can be adapted or configured for ELISA-based point-of-care testing. The kit can also include a second agent reactive with another biomarker, such as CA125, and at least one second reagent suitable for detecting expression of the other biomarker.

In another aspect, the present application provides a diagnostic kit for assessing risk of ovarian cancer in a female subject by measuring a level of Arresten expression in a biological sample obtained from the subject. The kit can include: (a) a capture antibody that is capable of specifically binding to human Arresten, thereby isolating human Arresten from the biological sample; (b) a solid matrix to which the capture antibody will bind; (c) a detection antibody that is capable of specifically binding to human Arresten and has a label for generating a detectable signal; (d) a recombinant human Arresten standard for calibration; and (e) at least one reagent suitable for generating the detectable signal in cooperation with the label, thereby detecting a level of human Arresten in the biological sample.

In some embodiments, the diagnostic kit can also include: (a) a second capture antibody that is capable of specifically binding to human CA125, thereby isolating human CA125 from the biological sample; (b) a second detection antibody that is capable of specifically binding to human CA125, and has a second label for generating a second detectable signal; (c) a recombinant human CA125 standard for calibration; and (d) at least one second reagent suitable for generating the second detectable signal in cooperation with the second label, thereby detecting expression of human CA125. In this context, a predetermined cut-off value of the human Arresten can be, for example, 80-100 pg/ml, and a predetermined cut-off value for the human CA125 can be, for example, 35-100 U/ml.

In a further aspect, the present application provides a device for determining whether a subject has ovarian cancer, by detecting Arresten expression in a urine sample of the subject. By way of example, the device can include a housing, a matrix of absorbent material, and an immunoassay strip. In certain embodiments, the housing contains the matrix and the strip (e.g., at least partially coated with a labeled anti-Arresten antibody). In use, a urine stream or urine sample of the subject can contact the absorbent matrix or pad of the device, which draws the liquid by capillary action into the immunoassay strip. Exemplary absorbent materials for use in the device of the present invention include nitrocellulose, polysulfones, polycarboxylic acids, filter paper, and the like.

As discussed in further detail below, the present application identifies Arresten as a new biomarker for ovarian cancer, with a sensitivity and specificity that are comparable to the existing biomarkers (e.g., CA125, HE4). Also described are uses of an antibody specific to Arresten for the diagnosis, treatment, or prognosis of ovarian cancer in a subject.

Using the methods, kits, antibodies, and devices of the present invention, it is possible to detect ovarian cancer, particularly type II ovarian cancer, at early stages of the disease (e.g., stage II). Moreover, it is possible to screen asymptomatic women periodically for ovarian cancer, and to detect ovarian cancer in women with a broad range of unspecific symptoms of ovarian cancer.

Additional aspects of the present invention will be apparent in view of the description which follows.

### BRIEF DESCRIPTION OF THE FIGURES

Embodiments of the invention will now be described, by way of example only, with reference to the accompanying drawings, in which:

FIG. 1 shows plasma concentrations of Arresten (pg/ml) in hospital controls versus ovarian cancer patients;

FIG. 2 shows a receiver operating characteristic (ROC) analysis of Arresten levels in ovarian cancer cases; and

FIG. 3 shows ROC analyses of Arresten levels in ovarian cancer cases separated by stage.

### DETAILED DESCRIPTION OF THE INVENTION

Arresten is a protein segment from the C-terminal of the collagen IV alpha chain, released due to collagen IV breakdown during extracellular matrix exposure in metastasis, starting at stage II. Prior to the present invention, Arresten had not been investigated in the context of ovarian cancer and has never been considered as a potential serum biomarker for ovarian cancer. The existing literature did not support use of Arresten as an ovarian cancer biomarker. The inventor's results, as disclosed herein, now identify Arresten as a new biomarker for diagnosis of ovarian cancer, especially ovarian cancer in stage II and higher.

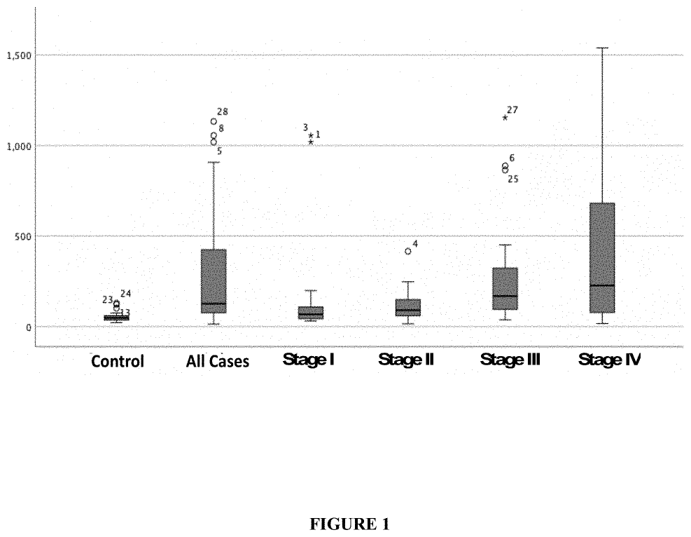

More specifically, the inventor demonstrates herein a significant increase of Arresten expression in diagnostic samples from ovarian cancer patients, as compared with healthy controls (see, e.g., FIG. 1). Accordingly, in one aspect, the present invention provides methods for determining whether a subject has ovarian cancer. The methods can include assaying a diagnostic sample of the subject for expression of Arresten, where detection of Arresten expression elevated above normal is diagnostic of neoplasia, particularly ovarian cancer, in the subject.

The Arresten biomarker disclosed herein can be used in methods for the diagnosis, prognosis, treatment, and monitoring of cancer, particularly ovarian cancer. In some embodiments, the methods of the present invention can be used to discriminate between healthy subjects and cancer subjects, including subjects with early-stage (e.g., stage II) disease. The methods can be based on the early detection, identification, or quantification of the Arresten biomarker, which is particularly well-suited to discriminate between healthy subjects and ovarian cancer subjects. The cancer subjects can include asymptomatic subjects and/or those at an early stage of the disease.

As a biomarker of ovarian cancer, Arresten can be detected in a biological sample, either alone or in combination with additional known biomarkers. By way of example, the biomarker can be detected, identified, or quantified by a screening method using biological or diagnostic samples from subjects. For instance, the biomarker can be detected in a blood or urine test. Known biomarkers of ovarian cancer include, without limitation, Cancer Antigen 125 (CA125) and Human Epididymis Protein 4 (HE4). Detection of Arresten expression in conjunction with detection of CA125 and/or HE4 can be particularly useful in the early detection of ovarian cancer and may significantly improve the accuracy of detecting pre-malignant changes or early-stage ovarian cancers in asymptomatic women at increased risk for the development of ovarian cancer.

Moreover, as a biomarker of ovarian cancer, Arresten can be detected and quantified in a biological sample in conjunction with other diagnostic techniques. For example, a test based on Arresten as a biomarker can be used in conjunction with vaginal examination, ultrasound, or MRI to diagnose ovarian cancer.

As noted above, ovarian cancer is asymptomatic in the early stages and most patients present with advanced levels of the disease. Cost-effective and non-invasive methods that can promote frequent testing may achieve early detection and high survival rates in ovarian cancer patients. Accordingly, in some embodiments of the methods described herein, Arresten can be detected through non-invasive tests. For example, subjects can be screened for Arresten expression using a simple and inexpensive detection module based on the well-known enzyme-linked immunosorbent assay (ELISA). This option can be useful for detecting Arresten in urine samples from subjects, including ovarian cancer patients.

Other objects, features, and advantages of the invention will become apparent from the following discussion. It should be understood, however, that the specific examples and preferred embodiments of the invention described herein are given by way of illustration only, and various changes and modifications within the scope of the invention will become apparent to those skilled in the art from the description which follows.

### Definitions

The following definitions are presented as an aid to understand the invention.

The term “subject” as used herein refers to a mammal, including, without limitation, a cow, dog, human, monkey, mouse, pig, or rat. Preferably, the subject is a human. More preferably, the subject is a woman.

The terms “sample”, “biological sample”, “diagnostic sample”, and the like, as used herein, refer to a material known or suspected of expressing or containing one or more cancer markers. The diagnostic sample can include any bodily fluids, tissues, or cells (e.g., blood, serum, plasma, urine, saliva, ovary tissues, mammary tissues, etc.). The sample is preferably a bodily fluid sample, such as blood, serum, plasma, vaginal secretions, urine, tears, saliva, etc.

As used herein, “blood” or “blood sample” can include a sample of whole blood, serum, or plasma, unless a different meaning is specified.

The terms “cancer” and “neoplasm” refer to a proliferation of tumour cells in tissue having the unique trait of loss of normal controls, resulting in unregulated growth, lack of differentiation, local tissue invasion, and/or metastasis, and include malignant tumours that are either invasive or non-invasive.

The phrase “primary cancer” refers to a cancer that is at a location of the body or a tissue where the particular cancer starts. Primary cancer is the opposite of metastasis, which refers to the migration of cancer cells from the original tumour site to produce cancer in other tissues. For example, a cancer originating in the ovary is called a “primary ovarian cancer”. If it metastasizes and spreads to the liver, the cancer is considered a primary ovarian cancer metastatic to the liver.

The terms “type I”/“subtype I” and “type II”/“subtype II” are used herein to refer to cancers, particularly ovarian cancers, which have remarkably different molecular genetic features as well as morphologic differences. For example, high-grade serous carcinoma (type II) is characterized by very frequent TP53 mutations, but rarely has mutations that characterize most type I carcinomas, including KRAS, BRAF, ERBB2, PTEN, CTNNB1, and PIK3CA. In general, type I tumours are genetically more stable than type II tumours and display a distinctive pattern of mutations that occur in specific cell types. Type II tumours which show greater morphologic and molecular homogeneity are genetically unstable and have a very high frequency of TP53 mutations. Therefore, it has been suggested that these two different types of ovarian cancers develop along different molecular pathways.

The term “marker” or “biomarker” refers to an indicator which can be detected in a sample, and includes predictive, diagnostic, and prognostic indicators and the like. The biomarker can be an indicator of a particular disease or disorder (e.g., ovarian cancer or other cancer), having certain molecular, pathological, histological, and/or clinical features.

The “presence”, “amount”, “concentration”, or “level” of a marker associated with an increased clinical benefit or disadvantage to an individual includes a detectable level of the marker in a sample. The presence, amount, or level of a marker can be measured by methods known to a person skilled in the art. Furthermore, the presence, amount, or level of a marker can be measured prior to treatment, during treatment, after treatment, or a combination of any of the foregoing.

As used herein in connection with Arresten, the term “expression” includes, without limitation, the transcription of the Arresten-associated gene into at least one mRNA transcript and/or the translation of at least one mRNA transcript into an Arresten protein. Accordingly, a diagnostic sample can be assayed for Arresten expression by assaying for Arresten, Arresten cDNA, or Arresten mRNA. The appropriate form of Arresten will be apparent based on the particular techniques discussed herein.

The phrase “elevated above normal”, as used herein, refers to expression of Arresten that is detected at a level significantly greater than the level expected for the same type of diagnostic sample taken from a non-diseased subject or patient (i.e., one who does not have cancer, such as ovarian cancer) of the same gender and of similar age. As further used herein, “significantly greater” refers to a statistically significant difference between the level of Arresten expression elevated above normal and the expected (normal) level of Arresten. Preferably, Arresten expression that is elevated above normal is expression of Arresten at a level that is at least 10% greater than the level of Arresten expression otherwise expected. Where Arresten expression is expected to be absent from a particular diagnostic sample taken from a particular subject or patient, the normal level of Arresten expression for that subject or patient is nil. Where a particular diagnostic sample taken from a particular subject or patient is expected to have a low level of constitutive Arresten expression, that low level is the normal level of Arresten expression for that subject or patient.

A “reference sample” or “control sample”, as discussed herein, is a biological sample provided from a reference or control group of apparently healthy individuals for the purpose of evaluation in vitro. Similarly, the expressions “reference concentration”, “reference value”, and “reference level”, as used herein, refer to a value established in a reference or control group of apparently healthy individuals. Determination of the reference concentration of Arresten or Arresten expression can be made based on an amount or concentration which best distinguishes patient and healthy populations. By way of example, the value for Arresten as determined in a control group or a control population establishes a “cut-off value” or a “reference range”. A value above such cut-off or threshold, or outside the reference range at its higher end, is considered to be “elevated above normal” or “diagnostic of ovarian cancer”. The reference level can be a single number, equally applicable to every subject, or the reference level can vary, according to specific subpopulations of subjects. For example, post-menopausal subjects can have a different reference level for ovarian cancer than pre-menopausal subjects. In addition, a subject with more advanced ovarian cancer (e.g., stages II-IV) can have a different reference value than one who has early stage ovarian cancer (e.g., stage I).

As used herein, an agent “reactive” with Arresten is one that has affinity for, binds to, or is directed against Arresten. Such an “agent” can be a protein, polypeptide, peptide, nucleic acid (including DNA or RNA), antibody, Fab fragment, F(ab′)₂ fragment, molecule, compound, antibiotic, drug, or any combination thereof. A Fab fragment is a univalent antigen-binding fragment of an antibody, which is produced by papain digestion. A F(ab′)₂ fragment is a divalent antigen-binding fragment of an antibody, which is produced by pepsin digestion. Preferably, the agent of the present invention is labeled with a detectable marker or label.

The term “antibody”, as used herein, refers to a specific protein molecule directed against an antigenic site and broadly includes all different types of antibody structures, such as monoclonal antibodies, polyclonal antibodies, multi specific antibodies (including bispecific antibodies), chimeric antibodies, humanized antibodies, fragments having antigen-binding activity, etc. For purposes of the present invention, the antibody can specifically bind to the biomarkers of the present invention, or the constituent proteins of the biomarkers, and can include polyclonal antibodies, monoclonal antibodies, and recombinant antibodies. The production of antibodies—using, for example, Arresten as an antigen—can be performed with techniques well known to a person of ordinary skill in the art.

The term “label” refers to a detectable compound or composition and “labelling” refers to the conjugation, fusion, or attachment of a detectable compound or composition to another. In some embodiments, the label is conjugated or fused directly or indirectly to an agent or reagent, such as an antibody, and assists with the detection of the agent to which it is conjugated or fused. The label itself can also be detectable (such as radioisotope labels or fluorescent labels and the like). By way of example, the label can be an enzymatic label which catalyzes chemical alteration of a substrate compound or composition and results in a detectable product.

The “sensitivity” of a biomarker, test, or assay, as described herein, means the probability that the biomarker, test, or assay will yield a positive result in an individual afflicted with cancer, particularly ovarian cancer. An increased sensitivity means fewer false negative test results. The “specificity” of a biomarker, test, or assay, as described herein, means the probability that the biomarker, test, or assay will yield a negative result in an individual not afflicted with cancer, particularly ovarian cancer. An increased specificity means fewer false positive test results.

The term “receiver operating characteristic curve” or “ROC curve” means a graphical plot that illustrates the diagnostic ability of a binary classifier system as its discrimination threshold or cut-off is varied. In common ROC curves, the true positive rate (TP=sensitivity) is plotted as a function against the false positive rate (FP=1−specificity) for different cut-off points for a particular biomarker or test. Each point on the ROC curve represents a specific sensitivity/specificity point that corresponds to a given threshold. The area under an ROC curve (AUC) can be a measure of how well a given biomarker or test can distinguish between two diagnostic outcomes. A threshold or cut-off value for a diagnostic test can be determined using ROC curve analysis. For example, the optimal cut-off value can be determined by giving equal weight to sensitivity and specificity with no ethical, cost, and prevalence constraints (e.g., Youden's index); alternatively, the optimal cut-off value can be determined by incorporating the costs of correcting false diagnosis and the costs of further work-up for diagnosis (e.g., utility-based decision theory). Therefore, if high costs are involved in false-positive diagnosis, the cut-off point can be selected at a higher value to maximize specificity, and if high costs are involved in false-negative diagnosis, the cut-off point can be selected at a lower value to maximize sensitivity.

The term “diagnosis”, as used herein, refers to the identification or classification of a molecular or pathological state, disease, or condition (e.g., cancer, a particular type of cancer, etc.) and broadly includes “screening”. “Diagnosis” also refers herein to the classification of a particular subtype of cancer, such as by histopathological criteria or by molecular features (including a subtype characterized by expression of one or a combination of biomarkers, such as particular genes or proteins encoded by the genes).

The term “prognosis”, as used herein, refers to the prediction of the likelihood of cancer-attributable death or progression, including recurrence, metastatic spread, and drug resistance, of a neoplasm, such as ovarian cancer. Prognosis may also be referred to in terms of “aggressiveness” or “severity”: an aggressive cancer is determined to have a high risk of negative outcome (i.e., negative or poor prognosis) and a non-aggressive cancer has a low risk of negative outcome (i.e., positive or favorable prognosis). An “aggressive” or “severe” tumour is a cell-proliferation disorder that has the biological capability to rapidly spread outside of its primary location or organ. Indicators of tumour aggressiveness that are standard in the art include, without limitation, tumour stage, tumour grade, Gleason grade, Gleason score, nodal status, and survival. In this context, the term “survival” is not limited to mean survival until mortality (wherein said mortality may be either irrespective of cause or related to a cell-proliferation disorder), but may also used in combination with other terms to define clinical outcomes (e.g., “recurrence-free survival”, in which the term “recurrence” includes both localized and distant recurrence; “metastasis-free survival”; “disease-free survival”, in which the term “disease” includes cancer and diseases associated therewith). The length of the survival may be calculated by reference to a defined starting point (e.g., time of diagnosis or start of treatment) and a defined end point.

The terms “treatment”, “treat”, “treating”, and “therapy” all refer to clinical intervention in an attempt to alter the natural course of an individual being treated, and can be performed either for prophylaxis or during the course of clinical pathology. Effects of treatment or therapy can include preventing occurrence or recurrence of disease, alleviating symptoms, diminishing any direct or indirect pathological consequences of disease, preventing metastasis, decreasing the rate of disease progression, ameliorating the disease state, minimizing the clinical impairment or symptoms resulting from the disease, diminishing any pain or discomfort suffered by the subject, remission or improved prognosis, and extending the survival of a subject beyond that which would otherwise be expected in the absence of such treatment. With reference to cancer, “treatment” and “therapy” also include inhibiting or preventing the development or spread of the cancerous cells (e.g., by limiting, suspending, terminating, or otherwise controlling the maturation and proliferation of cells involved in the cancer).

Ovarian Cancer and Diagnosis

According to conventional thought, low-grade serous carcinoma is a precursor lesion for high-grade serous carcinoma. However, recent studies support the inventor's conclusion that ovarian carcinoma has two subsets that are molecularly distinct as separate diseases characterized by differing patterns of genomic variation and prognostic implications. Low-grade serous carcinoma (e.g., classified as type I ovarian cancer) has better prognosis and a more indolent/stable genomic profile, while high-grade serous carcinoma (e.g., classified as type II ovarian cancer) has worse prognosis and an aggressive/distinct genomic profile.

Molecularly, type I ovarian cancer (OC) is often characterized by mutations in the mitogen-activated protein kinase (MAPK) pathway (e.g., KRAS or BRAF). Other significant variants for type I OC can include alterations of genes encoding β-catenin (e.g., CTNNB1), CDKN2A, PIK3CA, and PTEN which have also been found in a number of studies of type I OC. TP53 mutations have been rarely seen in type I OC, except in mucinous carcinoma.

Type II OC is characterized by a high degree of genomic/chromosomal instability, including nearly ubiquitous mutations in TP53 that arise mainly from the fallopian tube epithelium. The resulting dysfunctional p53 protein or pathway is the hallmark for the type II OC patient group. These tumours are clinically undetectable in stage I or II, and progress rapidly through stages III/IV.

Diagnosis of ovarian cancer can be performed when symptoms such as abdominal pain (bloating), gastrointestinal (e.g., bowel) irregularities), and pelvic pain (pressure or discomfort) are presented in a subject at advanced stages of the disease. 30% of patients experience a delay in diagnosis of 2-6 months. Prior to the present invention, OC has been diagnosed through a positive screening test, such as a CA125 test or transvaginal ultrasounds (TVS). Ovarian cancer can also be diagnosed incidentally after preventive surgery (i.e., for BRCA carriers).

Macroscopic residual disease after debulking surgery can be an independent prognostic factor for survival. Among women who had undergone primary debulking surgery, those with no residual disease had much better seven-year survival than women who had any residual disease (73.6% versus 21.0%; p<0.0001) [48]). Ten-year survival can be much improved when surgery results in no residual disease for stage III/IV HGSOC (50% versus 15% for any residual disease) [49].

In OC screening, the objective is to identify an ovarian cancer at a time when a cure is likely (e.g., when it is possible to achieve no residual disease after debulking). The likelihood of achieving no residual disease diminishes with the extent of abdominal disease the surgeon must remove. Therefore, among women with stage III ovarian cancer, it is desirable to diagnose cancer as early as possible with the goal of minimizing the intra-abdominal tumour burden. In one study, a status of no residual disease post-surgery was achieved for most patients with stage IIIa/b ovarian cancer, while only about 45% of patients with stage IIIc/IV ovarian cancer were reduced to no residual disease [48].

At present, 90% of patients are diagnosed in stage IIIc/IV; only 10% are diagnosed in stage IIIa/b. In this situation, a shift of patients from stage IIIc/IV to stage IIIa/b has the potential to increase the probability of cure. Currently, it is almost impossible to detect type II OCs at their stage I/II. Most stage I/II OCs that are diagnosed are type I OCs.

Existing Ovarian Cancer Biomarkers

To date, none of the known ovarian cancer serological biomarkers has been shown to be useful as a screening test in asymptomatic women because they have poor specificity and sensitivity in patients with early stage ovarian cancer. The only clinical significance of current ovarian cancer biomarkers is to differentiate between benign and malignant pelvic masses.

Current clinical guidelines recommend the use of biomarker CA125 for early triage of women with pelvic masses and for the management of patients with epithelial ovarian/fallopian tube cancer in monitoring response to first-line chemotherapy and post-therapy surveillance [10]. Concentrations of CA125 greater than 95 kU/L have discriminatory potential for malignancy in the pelvic mass, with a positive predictive value of 95% [11]. Persistent elevated levels of CA125 are indicative of a poor prognosis [12].

Nevertheless, as discussed above, the specificity and sensitivity of CA125 are far from ideal. Among epithelial ovarian cancer patients, only 80% or so have concentrations of CA125 above the reference interval of 35 kU/L. Elevations have been >90% in stages and 80-90% in stage II, but only 50-60% in patients with stage I OC [13,14].

Specificity of CA125 is compromised by its overexpression in healthy pre-menopausal women during menses and in pregnancy, as well as in some non-malignant gynecologic diseases (e.g., ovarian cysts, endometriosis, adenomyosis, uterine leiomyomas), peritoneal, pleural, and musculoskeletal inflammatory disorders, pelvic inflammatory diseases, and liver, renal, and cardiac diseases. Additionally, elevated concentrations can occur in most types of advanced adenocarcinomas, including breast, colorectal, pancreas, lung, endometrium, and cervix [14,15].

The frequency of CA125 overexpression is highest, though, in serous epithelial ovarian cancer patients, followed by endometrioid and clear cell types. CA125 is not expressed in pure mucinous histological type of epithelial ovarian cancer [14,15].

HE4 is also found to be overexpressed in ovarian cancer [16]. It has been shown to have greater specificity compared with CA125, especially in the pre-menopausal population, in which HE4 levels are less elevated than CA125 (8% versus 29% in benign pelvic mass); however, HE4 still shows varying results for sensitivity [17]. HE4 is not elevated in benign gynecological conditions (e.g., pregnancy, menstruation, endometriosis) [18].

HE4 demonstrates the highest sensitivity for stage I diagnosis (45.9% sensitivity at 95% specificity) and performs better than CA125 as an indicator of worse prognosis in epithelial ovarian cancer [19]. The HE4 serum levels in healthy women range from 60 pmol/L to 150 pmol/L, with higher serum levels observed in women over 40 years of age [20,21]. Concentrations of HE4 and CA125 are the highest in endometrioid cancer (100% overexpression) and serous epithelial ovarian cancer (93% overexpression), and the lowest in patients with mucinous ovarian carcinomas [22].

HE4 has also been identified in pulmonary, endometrial, and breast carcinomas, and mesotheliomas, but less frequently in gastrointestinal, renal, and transitional cell carcinomas. The most significant source of false-positive results in serum is renal failure [23].

In 2008, Moore et al. developed the Risk of Ovarian Malignancy Algorithm (ROMA) that uses a combination of CA125, HE4, and menopausal status to predict the presence of a malignant ovarian tumour with expected higher sensitivity and specificity compared with CA125 alone [19,24]. Several independent prospective studies and meta-analysis have been carried out in order to validate the diagnostic performance of ROMA, but they failed to reach a clear consensus.

Van Gorp et al. performed prospective validation on 389 patients and found that CA125 over-performed HE4 in post-menopausal women and neither HE4 nor ROMA improved the diagnosis of ovarian cancer [25]. There is some limited meta-analysis-based evidence of ROMA performing better in early ovarian cancer and the post-menopausal population. Other meta-analysis studies found ROMA to have higher sensitivity, but HE4 to be more specific. Overall, existing meta-analysis results do not provide strong evidence for ROMA superiority over CA125 alone [26-29].

In 2016, the United States Food and Drug Administration (FDA) approved a new-generation pre-operative serum biomarker test for ovarian cancer: the OVA1® test (Overa®). OVA1 combines 5 individual markers: CA125-II, HE4, apolipoprotein A-1, follicle stimulating hormone, and transferrin. Only after an ovarian mass has been determined to require surgery is this test then used to assess the likelihood of malignancy; thus, the use of the OVA1 test is very limited. OVA1 maintains a higher diagnostic sensitivity and high negative predictive value because five biomarkers are applied in parallel in the same serum specimen. If the test shows low-risk, the tumour is very unlikely to be malignant and the surgery can be scheduled without consulting a specialist [30].

In conclusion, CA125 is currently the only biomarker approved for routine use in ovarian cancer diagnosis. HE4, reporting increased specificity as compared to CA125, requires further validation of clinical utility. All of these biomarkers show elevation in the late-stage disease population, but it may not be clinically relevant. Since very high specificities and sensitivities are required in screening for diseases of low prevalence, neither CA125 nor HE4 qualifies as a screening marker.

Limits to Optimizing Cut-Off for Cancer Antigen 125

Cancer Antigen 125 (CA125) is expressed on the surface of ovarian cancer cells and plays a role in progression and metastasis of the disease. CA125 is also expressed in normal epithelia of the peritoneum, in endometrium, or in benign ovarian cysts. Various research has been conducted to assess the levels of CA125 in different population groups. It was reported that 1% of 883 healthy women, 6% of 143 patients with benign pelvic disease, and 82% of 102 patients with ovarian cancer showed levels of CA125 over 35 U/ml [50]. In addition, it was reported that 10% of benign pelvic disease (mostly pre-menopausal), 25% of borderline OC tumours, 40% of stage I OC tumours, and 80% of stage II-IV OC tumours showed levels more than 100 U/ml of CA125 [51].

It was previously reported that, among healthy individuals, 5% showed values of CA125 over 35 U/ml, 1% showed values over 65 U/ml, and 0.1% showed values over 100 U/ml (seen only in pre-menopausal women) [52]. More recently, Kotsopoulos et al. measured the levels of CA125 of 422 patients with ovarian cancers at different disease stages and obtained the following results [unpublished; included here with permission]:

| Stage | CA125, mean (range) | Median | CA125 >100, n (%)¹ | Missing/No CA125, n |
|---|---|---|---|---|
| IA (n = 47) | 424.2 (6.0-5642.0) | 73.0 | 16 (52%) | 8 |
| IB (n = 3) | 23.7 (9.0-42.0) | 20.0 | 0 | 0 |
| IC (n = 44) | 1013.6 (2.0-40542.0) | 54.0 | 15 (38%) | 5 |
| IIA (n = 17) | 224.9 (8.0-1298.0) | 114.0 | 10 (63%) | 1 |
| IIB (n = 28) | 556.3 (14.0-2912.0) | 184.0 | 16 (62%) | 2 |
| IIIA (n = 16) | 553.7 (15.0-2191.0) | 310.0 | 12 (80%) | 1 |
| IIIB (n = 45) | 1191.2 (27.0-12992.0) | 516.0 | 36 (84%) | 2 |
| IIIC (n = 145) | 1864.2 (2.0-26056.0) | 719.5 | 125 (87%) | 1 |
| IV (n = 57) | 1897.2 (54.0-11835.0) | 762.5 | 53 (98%) | 3 |
| Missing (n = 20) | 1871.2 (4.0-14450.0) | 199.0 | 13 (81%) | 4 |

As the above data show, if the cut-off value for CA125 is changed from 35 to 100 kU/L, more than 80% of ovarian cancer patients with stages III and IV will be identified; however, patients with stage I/II may not be identified.

As discussed above, the main limitations to the use of CA125 as a biomarker for ovarian cancer are its low sensitivity for stage I/II and its low specificity for pre-menopausal women. Currently, the recommended cut-off for CA125 is 35 U/ml internationally. Raising the cut-off to 100 U/ml could address both of these limitations, but at the expense of missing borderline and stage VII tumours, which are biologically low-risk and have a 10-year survival of over 80%.

Arresten in Oncogenesis

Type IV collagen proteins are major structural components of basement membranes. The general term “matrikines” has been employed for all collagen-derived fragments, out of which several are known to possess anti-angiogenic activity [42].

Arresten is the 26-kDa biologically active molecule and product of proteolytic cleavage derived from non-collagenous domain of type IV collagen, alpha-1 chain (encoded by the COL4A1 gene). The base sequence of the human COL4A1 gene and the amino acid sequence of the encoded protein are known. For example, the nucleotide sequence of the human COL4A1 gene and the protein amino acid sequence are registered and published in GenBank (GenBank Accession No. NM_001845). Arresten inhibits endothelial cell proliferation, at least in part, by inducing apoptotic mechanism [43].

Arresten as a Biomarker for Ovarian Cancer

The present application identifies Arresten as an ovarian cancer biomarker that has significantly discriminative power, without compromising on sensitivity, before manifestation of symptoms. Prior to the present invention, there was no study carried out regarding concentration of Arresten in blood, especially in connection with early detection or diagnosis of ovarian cancer. As detailed herein, the inventor found that Arresten is effective as a biomarker in ovarian cancer and its level can be used to distinguish an ovarian cancer patient from a normal subject.

As is well known in the art, reference levels of tumour markers initially arise from the need for a differentiation diagnosis between patients and healthy population. Similarly, serum levels of Arresten that differentiate ovarian cancer patients from healthy or hospital controls, who do not have ovarian cancer, can be determined. It is now possible to develop diagnostic methods and tools based on the use of Arresten as a biomarker for ovarian cancer.

The area under the receiver operating characteristic (ROC) curve, referred to as the AUC, can be considered an appropriate measure for describing the overall accuracy of a biomarker or a diagnostic test. In one embodiment, the AUC value of the Arresten biomarker for ovarian cancer, or a diagnostic method or test relating to same, is at least about 0.70, at least about 0.75, at least about 0.80, at least about 0.85, or at least about 0.90. In another embodiment, the sensitivity of the Arresten biomarker for ovarian cancer, or a diagnostic method or test relating to same, is at least about 60%, at least about 65%, at least about 70%, at least about 75%, at least about 80%, at least about 85%, or at least about 90%. In yet another embodiment, the specificity of the Arresten biomarker for ovarian cancer, or a diagnostic method or test relating to same, is at least about 75%, at least about 80%, at least about 85%, or at least about 90%.

A specific cut-off value for the Arresten biomarker for ovarian cancer, or a diagnostic method or test relating to same, can be determined for use in connection with individual subjects requiring diagnosis of ovarian cancer. In one embodiment, a threshold or cut-off value of Arresten that is determined to be elevated above normal (e.g., a reference concentration or cut-off value of Arresten) can be determined to be a value from 80 to 100 pg/ml. For example, the threshold value of Arresten can be one of the following concentrations in the subject's sample (e.g., serum or plasma sample): 80 pg/ml, 85 pg/ml, 90 pg/ml, 95 pg/ml, or 100 pg/ml. The reference concentration can also be selected from the ranges of 80-100 pg/ml, 85-100 pg/ml, 90-100 pg/ml, and 95-100 pg/ml. In certain embodiments, the reference concentration is further selected from the ranges of 80-100 pg/ml, 80-95 pg/ml, 80-90 pg/ml, and 80-85 pg/ml, or from the ranges of 80-100 pg/ml and 85-95 pg/ml. In another embodiment, the reference concentration can be at least two times the average concentration of Arresten in the samples of controls (e.g., serum or plasma samples). In yet another embodiment, the reference concentration can be 1.2-2 times, preferably, 1.2-1.5 times, the average concentration of Arresten in the samples of controls (e.g., serum or plasma samples).

In addition, the following factors can also be determined: (i) plasma levels of Arresten, CA125, and HE4 in ovarian cancer patients at the time of diagnosis and in healthy women matched for menopause status; (ii) sensitivity and specificity of Arresten in distinguishing ovarian cancer cases from healthy controls; and (iii) whether Arresten has a potential to enhance specificity and sensitivity of existing biomarkers, including CA125 and HE4, when used in combination with them.

As one preferred approach, Arresten can be combined with different biomarkers, such as CA125 and HE4, in the detection, diagnosis, monitoring, and prognosis of ovarian cancer. In particular, Arresten can address gaps in screening, early detection, and diagnosis of ovarian cancer. Since Arresten is cleared from blood by the kidney, a simple urine test for early detection of the disease can be provided for use by women in the comfort of their own homes (e.g., for use periodically or if they present with a symptom associated with ovarian cancer). As a consequence, Arresten as a biomarker can be used in screening methods and tools to screen the general population for ovarian cancer. Furthermore, interaction between p53 and Arresten can be characterized and quantified based on their expression levels in ovarian tumour tissues. Plasma levels of Arresten can be correlated with disease development in ovarian cancer patients, thereby providing new and non-invasive screening and diagnostic tests.

Diagnostic Samples

In accordance with the methods described herein, a diagnostic sample from the subject can be obtained using standard procedures. The diagnostic sample can be tissue and can be removed by standard biopsy. By way of example, the diagnostic sample can be any tissue known to have a neoplasm, any tissue suspected of having a neoplasm, or any tissue believed not to have a neoplasm. In addition, the diagnostic sample can be a bodily fluid, including blood, serum, plasma, vaginal secretions, urine, tears, and saliva.

Protein (e.g., Arresten) can be isolated and purified from the diagnostic sample of the present invention using standard methods known in the art, including, without limitation, extraction from a tissue (e.g., with a detergent that solubilizes the protein) where necessary, followed by affinity purification on a column, chromatography (e.g., FTLC and HPLC), immunoprecipitation (with an antibody to Arresten), and precipitation. Isolation and purification of the protein can be followed by electrophoresis (e.g., on an SDS-polyacrylamide gel). In accordance with the methods of the present invention, ovarian cancer in a subject can be diagnosed by assaying a diagnostic sample of the subject for expression of Arresten, wherein expression of Arresten elevated above normal is diagnostic of ovarian cancer.

Detecting Arresten Expression

In the methods of the present invention, a diagnostic sample of a subject can be assayed for Arresten expression, and Arresten expression can be detected in a diagnostic sample, using assays and detection methods readily determined from the known art (e.g., immunological techniques, hybridization analysis, fluorescence imaging techniques, and/or radiation detection, etc.), as well as any assays and detection methods disclosed herein (e.g., immunoprecipitation, Western blot analysis, etc.). For example, a diagnostic sample of a subject can be assayed for Arresten expression using an agent reactive with Arresten (e.g., antibody specific to Arresten).

The antibodies as used herein can be labeled with a detectable marker or label. Labeling of an antibody can be accomplished using one of a variety of labeling techniques, including a chemical (e.g., biotin), an enzyme (e.g., horseradish peroxidase, alkaline phosphatase), a radioactive material, a luminescent material, or a chemiluminescent material known in the art. For example, a nonradioactive or fluorescent marker, such as biotin, fluorescein (FITC), acridine, cholesterol, or carboxy-X-rhodamine, which can be detected using fluorescence and other imaging techniques readily known in the art. Alternatively, the detectable marker or label can be a radioactive marker, including, for example, a radioisotope. The radioisotope can be any isotope that emits detectable radiation, such as ³H, ¹⁴C, ³²P, ³⁵S, ⁴⁵Ca, or ¹²⁵I. Radioactivity emitted by the radioisotope can be detected by techniques well known in the art. For example, gamma emission from the radioisotope can be detected using gamma imaging techniques, particularly scintigraphic imaging. Preferably, the agent of the present invention is a high-affinity antibody labeled with a detectable marker or label.

Where the agent of the present invention is an antibody reactive with Arresten, a diagnostic sample taken from the subject can be purified by passage through an affinity column which contains Arresten antibody as a ligand attached to a solid support, such as an insoluble organic polymer in the form of a bead, gel, or plate. The antibody attached to the solid support can be used in the form of a column. Examples of suitable solid supports include, without limitation, agarose, cellulose, dextran, polyacrylamide, polystyrene, sepharose, or other insoluble organic polymers. The Arresten antibody can be further attached to the solid support through a spacer molecule, if desired. Appropriate binding conditions (e.g., temperature, pH, and salt concentration) for ensuring binding of the agent and the antibody can be readily determined by the skilled artisan. In one embodiment, the Arresten antibody is attached to a sepharose column, such as Sepharose 4B.

Where the agent is an antibody, a diagnostic sample of the subject can be assayed for Arresten expression using binding studies that utilize one or more antibodies immunoreactive with Arresten, along with standard immunological detection techniques. For example, the Arresten molecule eluted from the affinity column can be subjected to an ELISA analysis, Western blot analysis, flow cytometry, or any other immunostaining method employing an antigen-antibody interaction.

The detection of Arresten expression in the method of the present invention can be followed by an assay to measure or quantify the extent of Arresten expression in a diagnostic sample of a subject. Such assays are well known to one of skill in the art, and can include, without limitation, immunohistochemistry/immunocytochemistry, flow cytometry, mass spectroscopy, Western blot analysis, or an ELISA for measuring amounts of Arresten molecule. For example, to use an immunohistochemistry assay, histological (paraffin-embedded) sections of tissue can be placed on slides, and then incubated with an antibody against Arresten. The slides then can be incubated with a second antibody (against the primary antibody), which is tagged to a dye or other colorimetric system (e.g., a fluorochrome, a radioactive agent, or an agent having high electron-scanning capacity), to permit visualization of Arresten present in the sections.

It is contemplated that the diagnostic sample can be assayed for expression of any or all forms of Arresten protein (including precursor, endoproteolytically-processed forms, and other forms resulting from post-translational modification) in order to determine whether a subject or patient has ovarian cancer. It is also contemplated that the diagnostic sample may be assayed for expression of Arresten elevated above normal by detecting an increase in p53-Arresten interaction. Accordingly, in one embodiment of the present invention, Arresten expression elevated above normal is detected by detecting p53-Arresten interaction elevated above normal.

Expected or normal levels of Arresten expression for a particular diagnostic sample taken from a subject or patient can be determined by assaying non-diseased subjects of a similar age and of the same gender. For example, diagnostic samples can be obtained from at least 30 normal, healthy women within a certain age range (e.g., between the ages of 25 and 80), to determine the normal quantity of Arresten expression in females. Once the necessary or desired samples have been obtained, the normal levels of Arresten expression in women can be determined using a standard assay for quantification, such as flow cytometry, Western blot analysis, or an ELISA for measuring protein quantities.

By way of example, an ELISA can be run on each sample in duplicate, and the means and standard deviations of the quantity of the Arresten molecule can be determined. If necessary, additional subjects can be recruited before the normal quantities of Arresten expression are quantified.

Immunoassay Detecting Arresten

Immunoassays rely on antigen-antibody interactions, in which an antibody inherently binds to the specific structure of its associated molecule. Conventional immunoassay protocols can be adapted to detect Arresten as a biomarker to diagnose ovarian cancer in a subject. Suitable immunoassay methods include quantitative or qualitative immunoassay methods conventionally developed, including Western blotting (WB), immunofluorescence (IF), immunocytochemistry (ICC), immunohistochemistry immunoprecipitation (IP), enzyme-linked immunosorbent assay (ELISA), and flow cytometry (FCM).

All immunoassays employ a means to produce a measurable signal in response to antibody binding, and most involve chemically linking a detectable label to antibodies or antigens. Many labels are detectable because, for example, they emit radiation, produce a color change in a solution, fluoresce under light, or can be induced to emit light. Detectable signals can be measured by various detection methods, including colorimetry, immunofluorescence method, bioluminescence method, and chemiluminescence method.

Production of Anti-Arresten Antibody

In certain embodiments of the present invention, the agent reactive with Arresten is an antibody. Such an antibody can be produced using conventional methods known in the art, and can be polyclonal or monoclonal.

Polyclonal antibody can be produced, for example, by immunizing any animal species host, such as a rabbit, mouse, rat, sheep, goat, monkey, and the like, with purified Arresten. Monoclonal antibody then can be produced by removing the spleen from the immunized animal, and fusing the spleen cells with myeloma cells to form a hybridoma which, when grown in culture, will produce a monoclonal antibody (see, e.g., Kohler and Milstein [53]). Alternatively, a phage antibody library (see, e.g., Clackson et al. [54]; Marks et al. [55]) or any other methods known in the art may be used to produce monoclonal antibody. The antibodies of the present invention include functional fragments of antibody molecules. A “functional fragment” of an antibody is an antibody fragment having antigen-binding function.

In one embodiment, a diagnostic tool of the present invention uses, as a reagent, a monoclonal antibody specific to Arresten. Such an antibody can be produced by the following method: (i) polyclonal antibody is produced by immunizing a rabbit with purified human Arresten; (ii) monoclonal antibody is produced by removing the spleen from the immunized rabbit, and fusing the spleen cells with myeloma cells to form a hybridoma; and (iii) the hybridoma is grown in culture and the monoclonal antibody is isolated.

Types of ELISA and their Modes of Use

As noted above, Arresten expression in a biological sample can be detected and quantified by ELISA. In general, there are four types of ELISA: direct, indirect, sandwich, and competitive.

In a direct ELISA, Arresten is immobilized directly on a solid support (e.g., a microplate made of polystyrene or polyvinyl chloride) in a non-specific manner via adsorption to the surface. A detection antibody, which is conjugated with an enzyme (e.g., horseradish peroxidase (HRP), alkaline phosphatase (AP)), then binds to the immobilized Arresten, forming an antigen-antibody complex. Substrate for the enzyme (e.g., 3,3′5,5′-tetramethylbenzidine (TMB), para-nitrophenylphosphate (pNPP)) is then added, generating a signal that is proportional to the amount of Arresten in the sample. As an alternative, an amplified signal can be generated using a biotin-labelled detection antibody and an enzyme-conjugated protein that has an affinity to biotin, instead of the enzyme-conjugated detection antibody. For example, streptavidin and avidin are capable of forming a biotin complex because of their high affinity to biotin. While streptavidin lacks glycosylation, avidin is highly glycosylated. These proteins can be modified through glycosylation or deglycosylation.

In an indirect ELISA, Arresten is immobilized directly on a solid support in the same manner as in a direct ELISA, but the assay includes a further amplification step for detection. A primary detection antibody, which is unconjugated, is added and binds to Arresten; then a secondary detection antibody, which is conjugated with an enzyme, is added and binds to the primary antibody. Substrate for the enzyme is added and generates a signal proportional to the amount of Arresten in the sample.

In a sandwich ELISA, two antibodies specific to two different epitopes of Arresten are used to sandwich the protein. First, a capture antibody is coated on a solid support. Next, a biological sample containing Arresten is added, isolated by the capture antibody, and thereby specifically immobilized on the solid surface through the capture antibody. An enzyme-conjugated detection antibody is added and binds to an additional epitope on Arresten, thereby forming a complex of Arresten with the two antibodies. Substrate for the enzyme is added and generates a signal that is proportional to the amount of Arresten in the sample. Sandwich ELISAs are highly specific because two antibodies are required to bind to Arresten.

In the sandwich ELISA described above, the detection antibody may be unconjugated but can itself be detected by a secondary antibody that is conjugated to an enzyme, in the same manner as in a indirect ELISA. Another alternative can be to replace the enzyme-conjugated detection antibody with a biotin-labelled detection antibody and an enzyme-conjugated protein that has an affinity to biotin in the same manner as in the alternative mode of a direct ELISA.

In a competitive ELISA, a capture antibody is coated on a solid support in the same manner as in a sandwich ELISA. However, instead of using an enzyme-conjugated detection antibody, an enzyme-conjugated antigen is used to compete for binding with Arresten present in a sample. Substrate for the enzyme is added and generates a signal that is inversely proportional to the amount of Arresten in the sample.

Exemplary Sandwich ELISA and Protocol for Arresten

Commercially available antibodies or kits can be used in a sandwich ELISA. By way of example, antibodies and solutions for a sandwich ELISA can be obtained from R&D Systems, Inc. (e.g., Human Arresten DuoSet™ ELISA, catalog #DY9925-05, R&D Systems, Inc., Minneapolis, Minn., USA). Reagent diluents, wash buffers, or other solutions which could potentially impact assay performance can be routinely optimized based on type of sample (e.g., serum, plasma, etc.). An exemplary sandwich ELISA is described below.

Materials

A sandwich ELISA can include the following reagents: (a) murine capture antibody that is specific for human Arresten; (b) murine detection antibody that is biotin-labelled and specific for human Arresten; (c) a human Arresten standard for calibration (e.g., a purified recombinant human Arresten expressed in NS0 cell line); and (d) HRP-conjugated streptavidin. The assay is calibrated against the human Arresten standard.

The sandwich ELISA can also include: (i) 96-well microplates; (ii) plate sealers; (iii) phosphate-buffered saline (PBS) (137 mM NaCl, 2.7 mM KCl, 8.1 mM Na₂HPO₄, 1.5 mM KH₂PO₄, pH 7.2-7.4, 0.2 μm filtered); (iv) wash buffer (0.05% Tween® 20 in PBS, pH 7.2-7.4); (v) reagent diluent (5% Tween 20 in PBS, pH 7.2-7.4, 0.2 μm filtered); (vi) substrate solution (1:1 mixture of H₂O₂ and TMB); (vii) stop solution (2 N H₂SO₄).

Preparation of Reagents

To prepare working dilutions, all four reagents are brought to room temperature before use and allowed to sit for a minimum of 15 minutes with gentle agitation after initial reconstitution. 2.0 ml of HRP-conjugated streptavidin is diluted to a predetermined working concentration using reagent diluent. The capture antibody is reconstituted with 0.5 mL of PBS and diluted in PBS to a predetermined working concentration. The detection antibody is reconstituted with 1.0 mL of reagent diluent and diluted in the same to a predetermined working concentration. The recombinant human Arresten standard is reconstituted with 0.5 mL of reagent diluent for assay calibration.

Preparation of Microplates

The 96-well microplates in the sandwich ELISA are coated with 100 μL per well of the diluted capture antibody, sealed, and incubated overnight at room temperature. Each well of the microplates is then aspirated and washed with wash buffer and the process is repeated twice for a total of three washes. Each well is washed by filling with wash buffer (400 μL), with liquid being completely removed at each step. After the last wash, any remaining wash buffer is removed. The washed microplates are then blocked by adding 300 μL of reagent diluent to each well and incubated at room temperature for a minimum of an hour. The previous aspiration/wash step is repeated.

Assay Procedure

100 μL of each sample or standard diluted in reagent diluent is added to each well and the plates are sealed and incubated for 2 hours at room temperature. Each well of the microplates is aspirated and washed with wash buffer and the process is repeated twice for a total of three washes. Each well is washed by filling with wash buffer (400 μL), with liquid being completely removed at each step. After the last wash, any remaining wash buffer is removed. 100 μL of the detection antibody diluted in reagent diluent is then added to each well and the plates are sealed and incubated for 2 hours at room temperature. The previous aspiration/wash step is again repeated. 100 μL of the working dilution of HRP-conjugated streptavidin is then added to each well and the plates are covered and incubated for 20 minutes at room temperature. The previous aspiration/wash step is again repeated. 100 μL of substrate solution is added to each well and the plates are incubated for 20 minutes at room temperature. 50 μL of stop solution is then added to each well. The optical density of each well is immediately determined using a microplate reader set or corrected to one of the following wavelengths: 450 nm, 540 nm, or 570 nm.

A standard curve (calibration curve) is created based on a calibration data set for each set of samples assayed using the standard reagent (calibrator) prepared earlier. The calibration data set is generated at multiple concentrations or levels to assess amount, concentration, or level of Arresten in the sample.

For calculation of results, the duplicate readings for each standard and sample are averaged and the average zero standard optical density is subtracted from the averaged duplicate readings. Based on the foregoing, a standard curve is generated to obtain the concentration read for each sample.

Tests and Kits

The inventor's determination that an elevated level of Arresten expression can be detected in diagnostic samples from ovarian cancer patients provides new options for identifying patients with ovarian cancer, with the potential for commercial application in the form of diagnostic tools (e.g., tests, kits, etc.) for the diagnosis of ovarian cancer and for use in general screening procedures. Screening procedures can assist in the early detection and diagnosis of ovarian cancers, and can provide a method for the follow-up of patients in whom Arresten expression elevated above normal has been detected.

Accordingly, the present invention further provides tests and kits for use as assays for ovarian cancer. In some embodiments, the test or kit includes an agent reactive with Arresten and reagents suitable for detecting expression of Arresten. The agent can be any of those described above, and may be used in any of the above-described assays or methods for detecting or quantifying Arresten expression. Preferably, the agent of the present invention is labeled with a detectable marker or label.

As noted above, ELISA is one of several immunoassay methods used to detect and quantify specific target molecules. Three common types of ELISA are sandwich assays, competitive assays, and antigen-down assays. Sandwich assays are most commonly used because they deliver more sensitive and robust results.

In one embodiment, the diagnostic tool of the present invention is a sandwich ELISA capable of detecting Arresten in biological samples (e.g., blood, plasma, urine, etc.). By way of example, such a sandwich ELISA can include the following steps: (i) coating an antibody capable of binding to an epitope of Arresten, for use as a capture antibody attached to the surface of a solid substrate; (ii) reacting the capture antibody with the biological sample; (iii) reacting the resultant product of step (ii) with a detection antibody that is capable of binding to the complex of Arresten and the capture antibody and is labeled with a signal generating label; and (iv) measuring a signal originating from the label. The signal outputs can be measured in accordance with various methods known in the art. This detection of the signal enables a qualitative or quantitative analysis of the biomarker. If biotin is used as a label, it can be easily detected by streptavidin. When luciferase is used, luciferin can easily detect a signal. By analyzing the intensity of the final signal by the above-described immunoassay, ovarian cancer can be diagnosed. Specifically, when the signal for the biomarker in the biological sample of a subject appears stronger than in the normal sample, it can be determined that the subject has ovarian cancer.

In another embodiment, the diagnostic tool of the present invention is a compact point-of-care (POC) instrument employing ELISA for measuring Arresten in blood, plasma, serum, urine, and the like. The ELISA-based point-of-care (POC) testing may be suitable for use in resource-limited settings (e.g., a clinician's office) without requiring patients to visit a specialized laboratory for testing. The POC testing may adopt principles such as simplification of the procedures and miniaturization of the testing devices. Such a POC platform may, for example, combine the process of sandwich ELISA and the readout into a single microfluidic chip or cartridge, thereby providing a fully integrated, instrument-free, low-cost, and portable device for OC screening. Antibodies with relatively high affinity to Arresten may be preferred for such POC testing methods.

Due to its easy access, a POC test for ovarian cancer can be more readily available for screening of women with any relevant symptoms. Especially when symptoms are vague at early stages of the disease, availability of such convenient diagnostic tools may change the behaviors of clinicians and their patients, and result in promotion of routine screening of the population for ovarian cancer.

In certain embodiments, POC technology is combined with a urine test. Urine testing is particularly advantageous because urine samples are easily available and can be collected frequently in a non-invasive way. Correlation between urine and blood levels of Arresten can demonstrate the usefulness of urine detection of Arresten as a surrogate for detection of Arresten in plasma.

By way of example, a subject's urine sample may be screened for Arresten levels as part of a routine health examination. The test may be formatted as a urine test strip (e.g., as a standalone strip, cassette, or dipstick for laboratory use). A urine test strip for identifying whether a subject is at risk of having or has ovarian cancer may comprise a reagent that provides a response to the presence of Arresten when immersed in, and removed from, a urine sample of the subject; such a response indicates whether the subject is at risk of having or has ovarian cancer.

The urine test strip may be produced by means known to a person skilled in the art. In one embodiment, the urine test strip is provided in a device for testing a urine sample of a subject. The urine test strip may be prepared with a pad or matrix of an absorbent material. A labeled antibody specific to Arresten may also be provided in an area on the urine strip, so that urine, the labeled antibody, and Arresten flow together through the absorbent material by capillary action. The strip can be prepared from any suitable material through which the urine test sample, any Arresten therein, and labeled antibody can flow by capillarity. Suitable matrix materials include, without limitation, nitrocellulose, polysulfones, polycarboxylic acids, and filter paper.

In another embodiment, a urine test device may contain an immunoassay strip, an absorbent matrix or pad, and a plastic housing. The immunoassay strip can be formed by compressing nonwoven fibers into a narrow strip and coating them at least partially with a reactive antibody. In use, the antibody of the device may bind to Arresten present in a urine sample, ultimately resulting in a color change. The absorbent pad or matrix may extend to contact the urine stream of a user. The absorbent matrix can absorb the liquid and draw it into contact with the immunoassay strip. The immunoassay strip and the absorbent pad may be contained within a 2-piece housing that allows the unit to be handheld and protects the strip from environmental contaminants. A leak-proof, clear plastic window on the side of the housing can prevent urine from accidentally splashing on the immunoassay strip and permit the test and control zone portions of the strip to be viewed.

In still another embodiment, a strip-based ELISA test, similar to a pregnancy test, can be used by individual women at their own homes to detect Arresten in urine samples. This can promote ovarian cancer screening for even asymptomatic women, obviating the need to go to a doctor's office or laboratory for testing.

As noted above, ovarian cancers are associated with high fatality, but early detection can significantly improve survival rates. Convenient urine tests for use in detecting Arresten as a biomarker in ovarian cancer may promote early detection in patients, thereby increasing survival rates.

Exemplary Methods

In accordance the present invention, Arresten as an ovarian cancer biomarker can be detected, alone or in combination with other markers such as CA125 and HE4, in a biological sample. As discussed above, the cut-off value for CA125 can be increased (e.g., to 95-100 U/ml) in order to increase sensitivity of the biomarker for late-stage ovarian cancer (e.g., stage III or IV), but at the expense of detecting patients with early stage ovarian cancer (e.g., stage I or II). Combining Arresten detection with CA125 detection (with an increased cut-off value) may compensate for detecting stage I/II patients. Therefore, in one embodiment, a diagnostic sample of a subject is assayed for detecting the expression of both Arresten and CA125 to diagnose ovarian cancer in the subject. In certain preferred embodiments, the cut-off value for CA125 is 80-120 kU/L, 85-110 kU/L, 90-105 kU/L, or 95-100 kU/L. More preferably, the cut-off value for CA125 is 95 kU/L or 100 kU/L.

The present invention further provides a method for assessing the efficacy of therapy to treat ovarian cancer in a subject or patient who has undergone or is undergoing treatment for ovarian cancer. The method includes assaying a diagnostic sample of the subject or patient for Arresten expression, where detection of a normal level of Arresten expression is indicative of successful therapy to treat ovarian cancer, and detection of Arresten expression elevated above normal is indicative of not responding to treatment. The diagnostic sample can be a tissue or a bodily fluid, as described above, and can be assayed for expression of Arresten. In addition, the diagnostic sample can be assayed for expression of Arresten using all of the various assays and methods of detection and quantification described above.

This method of the present invention provides a means for monitoring the effectiveness of therapy to treat ovarian cancer by permitting the periodic assessment of levels of Arresten expression in a diagnostic sample taken from a subject or patient. In accordance with this method, a diagnostic sample of a subject or patient can be assayed, and levels of Arresten expression can be assessed, at any time following the initiation of therapy to treat ovarian cancer. For example, levels of Arresten expression can be assessed while the subject or patient is still undergoing treatment for ovarian cancer. Where levels of Arresten expression detected in an assayed diagnostic sample of the subject or patient continue to remain elevated above normal, a physician can choose to continue with the subject's or patient's treatment for the cancer or change it to a treatment to which tumour cells better respond. Where levels of Arresten expression in an assayed diagnostic sample of the subject or patient decrease through successive assessments, it can be an indication that the treatment for ovarian cancer is working, and that treatment doses could be decreased or even ceased. Where levels of Arresten expression in an assayed diagnostic sample of the subject or patient do not rapidly decrease through successive assessments, it can be an indication that the treatment for ovarian cancer is not working, and that treatment doses could be increased or the choice of treatment could be changed.

It is within the confines of the present invention to assess levels of Arresten expression following completion of a subject's or patient's treatment for ovarian cancer, in order to determine whether the cancer has recurred in the subject or patient. Accordingly, an assessment of levels of Arresten expression in an assayed diagnostic sample can provide a convenient way to conduct follow-ups of patients who were previously diagnosed with ovarian cancer. Furthermore, it is within the confines of the present invention to use assessed levels of Arresten expression in an assayed diagnostic sample as a clinical or pathologic staging tool, as a means of determining the extent of ovarian cancer in the subject or patient, and as a means of ascertaining appropriate treatment options.

A correlation exists, in general, between tumour burden and the survival of a patient who has cancer, and, more specifically, between pelvic mass and an ovarian cancer patient. Therefore, it is also contemplated herein that assaying a diagnostic sample of a subject for Arresten expression can be a useful means of providing information concerning the prognosis of a subject or patient who has ovarian cancer. Accordingly, the present invention further provides a method for assessing the prognosis of a subject who has ovarian cancer, comprising assaying a diagnostic sample of the subject for Arresten expression, where the subject's prognosis improves with a decrease in Arresten expression in the diagnostic sample of the subject, and the subject's prognosis worsens with an increase in Arresten expression in the diagnostic sample of the subject.

According to the method of the present invention, a diagnostic sample of a subject or patient can be assayed, and levels of Arresten expression can be assessed, at any time during or following the diagnosis of ovarian cancer in the subject or patient. For example, levels of Arresten expression in an assayed diagnostic sample can be assessed before the subject or patient undergoes treatment for ovarian cancer, in order to determine the subject's or patient's initial prognosis. Additionally, levels of Arresten expression in an assayed diagnostic sample can be assessed while the subject or patient is undergoing treatment for ovarian cancer, in order to determine whether the subject's or patient's prognosis has become more or less favorable through the course of treatment.

By way of example, where levels of Arresten expression detected in an assayed diagnostic sample of the subject or patient are, or continue to remain, significantly high, a physician can conclude that the subject's or patient's prognosis is unfavorable. Where Arresten expression in an assayed diagnostic sample of the subject or patient decreases through successive assessments, it can be an indication that the subject's or patient's prognosis is improving. Where levels of Arresten expression in an assayed diagnostic sample of the subject or patient do not decrease significantly through successive assessments, it can be an indication that the subject's or patient's prognosis is not improving. Finally, where Arresten expression is low, or is normal, in a diagnostic sample of the subject or patient, a physician can conclude that the subject's or patient's prognosis is favorable.

The present invention also provides methods of treating a subject who has been diagnosed with ovarian cancer using Arresten as a biomarker. The standard treatment for ovarian cancer currently consists of debulking surgery followed by six rounds of chemotherapy. In one embodiment, a diagnostic sample of a patient is analyzed for Arresten expression, and a therapy is provided to the subject when the Arresten expression is elevated above normal. Exemplary therapies include, without limitation, surgical debulking, chemotherapy, or radiation therapy, and any combination of the foregoing, including the current standard treatment of debulking surgery followed by chemotherapy. Typical chemotherapy drugs combine a platinum-based drug (e.g., carboplatin or cisplatin) with a taxane (e.g., paclitaxel or docetaxel).

The present invention is described in the following Examples, which are set forth to aid in an understanding of the invention, and should not be construed to limit in any way the scope of the invention as defined in the claims which follow thereafter.

### EXAMPLES

### Example 1—Study Subjects I

Plasma samples derived from ovarian cancer patients (n=22) and healthy controls (n=20) were tested for Arresten levels by an enzyme-linked immunosorbent assay (ELISA).

The ovarian cancer cases were obtained from the Poland Ovarian Cancer Study (POC). The POC study consisted of women with familial ovarian cancer who were referred to 1 of 16 clinical cancer genetics centers throughout Poland between 1999 and 2001. These centers were established in 1998 as a national network with support from the Polish Ministry of Health for the purpose of coordinating cancer genetics services. Eligible women included those with invasive ovarian cancer and at least one first- or second-degree relative with ovarian cancer diagnosed at any age or with early-onset breast cancer (diagnosed at age 50 or below). Of those, 22 ovarian cancer patients with high-grade serous type were randomly selected for this study before they started treatment.

The control women were those who participated in mammography screening for their routine screening tests at 8 different centers all over Poland between 2009 and 2011 and who provided blood samples for DNA analysis. Women with breast or ovarian cancer were excluded from this group; 20 healthy women were randomly selected for the study.

### Example 2—Laboratory Assay I

Plasma Arresten was quantified using a laboratory-made ELISA. All plasma samples were run in duplicate. The Arresten concentration was calculated as the average of duplicate samples (each adjusted for background signal and normalized to blank wells). The average intra-assay coefficient of variation (CV) was approximately 1.7%. This was calculated using the mean CV of duplicate samples. A non-parametric Mann-Whitney U test was used for comparing the mean Arresten plasma levels in plasma of the cases and the controls.

### Example 3—Detection of Arresten in Urine Samples

The same ELISA method of Example 2 that was used for detecting Arresten levels in plasma was also used to measure Arresten in urine samples of four healthy individuals. It was shown that Arresten is detectable in urine and its urine level generally correlates with the matched plasma level.

A urine-based biomarker test facilitates screening and diagnosis processes due to non-invasiveness of the collection method and the feasibility of developing ELISA paper strips to be used by women periodically for testing for ovarian cancer in the comfort of their own homes.

### Example 4—Validation Study I

400 plasma samples are collected from ovarian cancer patients with different histology, and 400 control samples collected from healthy subjects. Ovarian cancer plasma samples are obtained from Princess Margaret Hospital's Ovarian Cancer Biobank in Toronto, Canada. The control plasma samples are obtained from the biobank at Women's College Hospital in Toronto. From each patient, 300 μl of plasma is used for measuring three biomarkers: Arresten, HE4, and CA125. Three sections (10 microns each) from ovarian-tumour formalin-fixed paraffin-embedded (FFPE) blocks are obtained from 20 randomly-selected patients for measuring p53 and Arresten expression in tumour tissues.

Arresten levels are determined by enzyme-linked immunosorbent assay (ELISA). The specificity and sensitivity of Arresten as a biomarker are determined, alone and in combination with HE4 and CA125—other known biomarkers which are also measured by ELISA.

The expression levels of Arresten and p53 in ovarian tumour tissues are measured by immunohistochemistry (IHC) assay.

In order to determine diagnostic performance and optimal cut-off values for Arresten plasma levels, the sensitivities, specificities, and area under a receiver operating characteristic curve (AUC) are calculated. Similar calculations are performed for CA125 and HE4 and for combined biomarkers. The significance of two-group comparisons are calculated using a Mann-Whitney non-parametric test. Biomarker composite scores are calculated by logistic regression from standardized biomarker values.

The power of this study is more than 90% to detect a 10% difference in mean of measured biomarkers between patient cases and controls, based on 400 samples in each group.

### Example 5—Study Subjects II

For Validation Study II (Example 7), the inventor examined plasma samples from 421 unselected ovarian cancer patients with different histology and 407 hospital control patients. The hospital controls were non-cancer patients with urinary incontinence (252) or myoma (153). Ovarian cancer and control plasma samples were obtained from hospitals in Szczecin, Poland affiliated with Pomeranian Medical School. These samples were collected from ovarian cancer patients at the time of diagnosis between 2019 and 2021. The controls were women referred to the hospitals for non-malignant issues. Controls matched cases for age and menopausal status. The individuals in the study consented to participate in a biobank for studying ovarian cancer. Patients' characteristics and clinical data were collected at the time of diagnosis.

### Example 6—Laboratory Assay II

Arresten levels in the study subjects of Example 5 were determined by sandwich ELISA (enzyme-linked immunosorbent assay). Human Arresten DuoSet™ ELISA (Catalog #DY9925-05) from R&D Systems, Inc. (Minneapolis, Minn., USA) was routinely optimized for plasma samples in the inventor's lab at Women's College Hospital and used in the study. All measurements were made in duplicate and samples with a coefficient of variability (CV) of more than 15% were excluded from the study.

### Example 7—Validation Study II

The plasma levels of Arresten were measured and compared among the study subjects (421 ovarian cancer cases and 407 hospital control women) of Example 5. The significance of two-group (cases and controls) comparisons were calculated using either the Mann-Whitney non-parametric test where the distributions were not normal or a Student's t-test where the distributions were normal. Adjustment for confounding variables and further analysis were conducted by logistic regression. Biomarker performance was determined by calculating the AUC—the area under the receiver operating characteristic (ROC) curve. The optimal cut-off, sensitivity, and specificity of the biomarker were also determined by analyzing the ROC curve.

As discussed in greater detail below, the data showed that plasma levels of Arresten were significantly higher in ovarian cancer patients than in controls and could have use as a biomarker for diagnosis and tumour staging of ovarian cancer. This study's power to detect a 10% difference in the mean of measured biomarkers between cases and controls based on over 400 samples in each group was more than 90%.

### RESULTS AND DISCUSSION

### Results and Discussion for Examples 1 and 2

The mean plasma level of Arresten was significantly higher in ovarian cancer patients than in healthy controls (1020 ng/ml versus 446 ng/ml; p=0.0001). This represents a two-fold higher plasma level among ovarian cancer patients, which is opposite to what was expected. The higher concentration of Arresten in the plasma of ovarian cancer patients makes this protein even more favorable as a screening or diagnostic biomarker, despite the contrary hypothesis that linked Arresten with defective p53 function in ovarian cancers. One explanation for the contrary observation could be that Arresten is expected to be triggered by high expression levels of p53 even in its mutated, non-functional form. Preferably, it could be determined whether ovarian cancer patients tested in Examples 1 and 2 also carry high expression levels of the mutant p53 protein.

### Results and Discussion for Examples 5-7

Table 1 summarizes the details of plasma levels of Arresten measured in the 421 ovarian cancer patients and the 407 hospital controls who were the study subjects of Example 5:

TABLE 1
Statistics of Arresten levels in control and case samples.

| | | | p-Value |
|---|---|---|---|
| Group | Mean (pg/ml) | Median (pg/ml) | (comparison with control) |
| Control | 66.27 | 57.76 | Reference |
| All Cases | 247.95 | 115.97 | <0.001 |
| Stage 1 | 89.31 | 61.07 | 0.5 |
| Stage II | 114.64 | 90.29 | <0.001 |
| Stage III | 258.07 | 170.46 | <0.001 |
| Stage IV | 526.99 | 350.69 | <0.001 |

As the results show, the ovarian cancer patients had a mean Arresten level of 247.95 pg/ml, which was significantly higher than the mean Arresten level of the controls (66.27 pg/ml; p<0.001) (see FIG. 1; Table 1).

Comparing the ovarian cancer patients with the controls at each tumour stage, the Arresten levels of patients with stage I tumours showed no significant difference with the hospital controls (mean: 89.31 pg/ml versus 66.27 pg/ml; p=0.5). However, plasma concentrations of Arresten in patients with stage II, III, or IV tumours were significantly higher than the hospital controls (mean for stage II=114.64 pg/ml, mean for stage III=258.07 pg/ml, mean for stage IV=526.99 pg/ml versus hospital controls=66.27 pg/ml; p<0.001) (see FIG. 1; Table 1).

With reference to FIG. 1, Arresten levels were significantly higher in the ovarian cancer cases than in the controls (p<0.001). When controlling for stage, Arresten levels of the ovarian cancer cases at stage I were not significantly different from the controls (p=0.5). However, the difference between cases and controls in stages II-IV was significant (p<0.001).

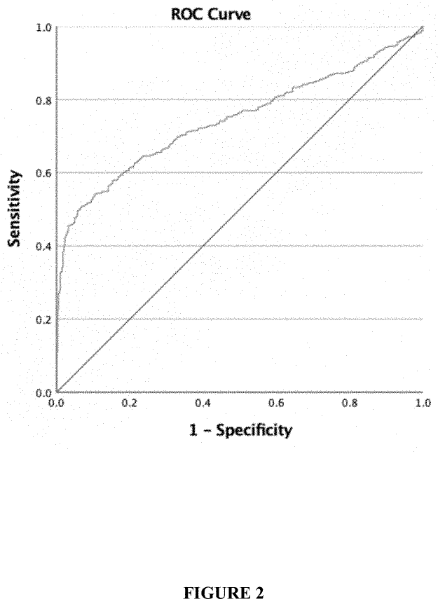

With reference to FIG. 2, analyzing the receiver operating characteristic (ROC) curve revealed an AUC of 0.745, with an optimal sensitivity of 64.6% and specificity of 76.63% at a cut-off value of 78.9 pg/ml (at specificity=90%, sensitivity=32.8%).

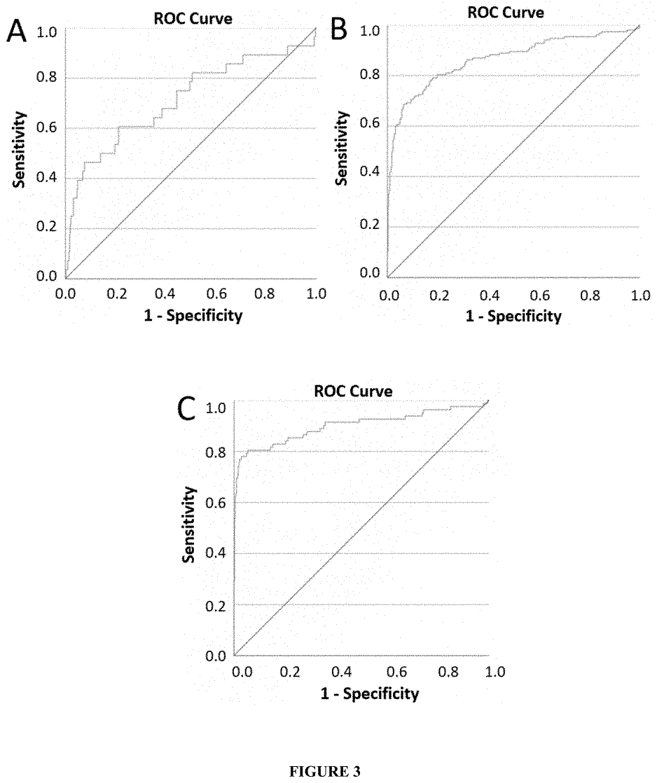

With reference to FIG. 3, the ROC curve for the stage II patients resulted in an AUC of 0.713, with an optimal sensitivity of 60.7% and specificity of 78.8% at a cut-off value of 81.88 pg/ml (at specificity=90%, sensitivity=7.1%) (see FIG. 3A). The ROC curve for the stage III patients resulted in an AUC of 0.865, with an optimal sensitivity of 80.4% and specificity of 80.2% at a cut-off value of 83.96 pg/ml (at specificity=90%, sensitivity=41.8%) (see FIG. 3B). The ROC curve for the stage IV patients resulted in an AUC of 0.900, with an optimal cut-off value of 121.75 pg/ml, sensitivity of 80.5%, and specificity of 94.6% (at specificity=90%, sensitivity=69.5%) (see FIG. 3C).

As the foregoing results reveal, Arresten plasma levels were higher in ovarian cancer cases than in hospital non-cancer controls (p<0.001), and levels increase with tumour stage. Arresten levels at stage I of ovarian cancer were not significantly different from controls. This was not unexpected, because Arresten is released due to extracellular matrix breakdown during metastasis—which, in the case of ovarian cancer, starts from stage II. However, after stage I, Arresten levels increased as cancer progressed through the various stages, and its performance as a diagnostic biomarker was improved in advanced stages, as shown by the increasing AUC of the ROC analysis of stages II to IV (see FIGS. 1 and 3).

Overall, Arresten showed a decent performance as an ovarian cancer biomarker with a sensitivity of 64.6% and specificity of 76.63% (see FIG. 2). Arresten performance was improved at advanced stages; however, its levels were significantly higher in ovarian cancer cases than in controls as early as stage II. These results support use of Arresten as a potential diagnostic biomarker for ovarian cancer, and use of plasma levels of Arresten as an indicator of tumour stage.

### ENUMERATED EMBODIMENTS

The invention is further exemplified by the following enumerated non-limiting embodiments that would be understood by those skilled in the art to be merely exemplary of the methods, uses, kits, and other implementations consistent with the description herein.

### Numbered Embodiments—Example A

1. A method for determining whether a subject has ovarian cancer, comprising assaying a diagnostic sample of the subject for Arresten expression, wherein detection of Arresten expression elevated above normal is diagnostic of ovarian cancer in the subject.
2. The method of embodiment 1, wherein Arresten expression elevated above normal is detected by detecting p53-Arresten interaction elevated above normal.
3. The method of embodiment 1, further comprising the step of obtaining the diagnostic sample from the subject.
4. The method of embodiment 1, wherein the ovarian cancer is an epithelial carcinoma.
5. The method of embodiment 4, wherein the epithelial carcinoma is a serous, mucinous, endometrioid, clear cell, transitional cell, squamous cell, or mixed epithelial neoplasm.
6. The method of embodiment 1, wherein the ovarian cancer is a p53-associated ovarian cancer.
7. The method of embodiment 1, wherein the ovarian cancer is primary ovarian cancer.
8. The method of embodiment 1, wherein the ovarian cancer is high-grade serous ovarian cancer.
9. The method of embodiment 1, wherein the diagnostic sample is a serum, plasma, or urine sample.
10. The method of embodiment 1, wherein the diagnostic sample is assayed using an agent reactive with Arresten.
11. The method of embodiment 10, wherein the agent is an antibody or an antigen-binding fragment thereof.
12. The method of embodiment 10 or 11, wherein the agent is labeled with a detectable marker.
13. The method of embodiment 1, wherein the diagnostic sample is assayed using an ELISA, a chemiluminescence assay, or an immunohistochemistry assay.
14. The method of embodiment 13, wherein the ELISA is an ELISA paper strip.
15. The method of embodiment 1, wherein the detected Arresten expression is at least two times higher than normal.
16. The method of embodiment 1, wherein the Arresten expression is elevated above normal in the diagnostic sample when the Arresten expression is above a concentration selected from 400 to 1000 ng/ml.
17. The method of embodiment 16, wherein the diagnostic sample is plasma.
18. The method of embodiment 1, wherein the subject is at least one of the following:

(a) pre-menopausal;

(b) asymptomatic of ovarian cancer;

(c) not carrying a mutation of BRCA1 or BRCA2 gene;

(d) suffering from a non-malignant gynecologic disease, a peritoneal, pleural, or musculoskeletal inflammatory disorder, a pelvic inflammatory disease, a liver, renal, or cardiac disease, or an advanced adenocarcinoma.

19. The method of embodiment 1, wherein the ovarian cancer is a type II ovarian cancer.
20. The method of embodiment 19, wherein the type II ovarian cancer is at stage I, II, Ma, or IIIb.
21. The method of embodiment 1, wherein the ovarian cancer is undetectable by a CA125 test or a transvaginal ultrasound test.
22. The method of embodiment 1, further comprising assaying the diagnostic sample of the subject for at least one additional biomarker selected from CA125 and HE4.
23. The method of embodiment 22, wherein the at least one additional biomarker is CA125 with a cut-off value of 95-100 U/ml.
24. A method of screening a general population for ovarian cancer, comprising: testing a plurality of asymptomatic subjects in accordance with the method of embodiment 1.
25. A method for treating ovarian cancer in a subject, comprising:

(a) analyzing a diagnostic sample of the subject for Arresten expression; and

(b) providing a therapy to the subject when the Arresten expression is elevated above normal,

wherein the therapy comprises at least one of surgical debulking, chemotherapy, radiation, and hormone therapy.

26. The method of embodiment 25, wherein the ovarian cancer is high-grade serous ovarian cancer.
27. The method of embodiment 26, wherein the high-grade serous ovarian cancer is at stage I, II, Ma, or IIIb.
28. A method for determining the molecular subtype of ovarian cancer in a patient who has been diagnosed with ovarian cancer, comprising:

(a) assaying a biological sample of the patient for Arresten expression; and

(b) either:
(i) determining that the patient has type I ovarian cancer when the Arresten expression in the biological sample is normal, or
(ii) determining that the patient has type II ovarian cancer when the Arresten expression in the biological sample is elevated above normal.
   29. The method of embodiment 28, wherein:

detection of Arresten expression elevated above normal is indicative of type II ovarian cancer in which p53 in ovarian cancer cells is mutated, and

detection of normal Arresten expression is indicative of type I ovarian cancer in which p53 in ovarian cancer cells is not mutated.

30. A method for assessing the severity of ovarian cancer in a patient who has been diagnosed with ovarian cancer, comprising:

(a) assaying a biological sample of the patient for Arresten expression prior to treatment; and

(b) either:
(i) determining that the patient has a favorable prognosis when Arresten expression in the biological sample is normal, or
(ii) determining that the patient has a poor prognosis when Arresten expression in the biological sample is elevated above normal.
   31. The method of embodiment 30, wherein:

detection of Arresten expression elevated above normal in the biological sample is indicative of ovarian cancer in which p53 in the ovarian cancer cells is mutated, and

detection of normal Arresten expression in the biological sample is indicative of ovarian cancer in which p53 in the ovarian cancer cells is not mutated.

32. A method for assessing the efficacy of therapy to treat ovarian cancer in a subject who has undergone or is undergoing treatment for ovarian cancer, comprising:

(a) assaying a first diagnostic sample of the subject for Arresten expression after therapy has commenced;

(b) obtaining a first level of Arresten expression in the first diagnostic sample; and

(c) comparing the first level of Arresten expression with a second level of Arresten expression in a second diagnostic sample of the same subject, wherein the second diagnostic sample was assayed and the second level was obtained prior to the therapy,

wherein a significant decrease of the first level of Arresten expression, relative to the second level of Arresten expression, indicates that the subject is responding to the therapy to treat ovarian cancer, and a minor or no decrease of the first level of Arresten expression, relative to the second level of Arresten expression, indicates that the subject is not responding to the therapy.

33. The method of embodiment 32, wherein the first level of Arresten expression is below a concentration selected from 400-1000 ng/ml, and the second level of Arresten expression is above the selected concentration.
34. A kit for use in detecting ovarian cancer, comprising:

(a) an agent reactive with Arresten; and

(b) at least one reagent suitable for detecting expression of Arresten.

35. The kit of embodiment 34, which is adapted for ELISA-based point-of-care testing.
36. The kit of embodiment 35, further comprising a second agent reactive with CA125 and at least one second reagent suitable for detecting expression of CA125.
37. The kit of embodiment 36, wherein:

the Arresten cut-off value is 400-1000 ng/ml, preferably 446-1020 ng/ml, and

the CA125 cut-off value is 95-100 U/ml.

38. A device for detecting ovarian cancer in a subject, comprising:

(a) a housing;

(b) a matrix of absorbent material for contacting a urine sample of the subject; and

(c) a strip of immunoassay at least partially coated with a labeled anti-Arresten antibody for detecting Arresten in the subject's urine sample,

wherein the absorbent matrix and the immunoassay strip are arranged within the housing in a manner such that the absorbent matrix, upon contact with the urine sample, draws the liquid into the immunoassay strip by capillary action.

39. The device of embodiment 38, wherein the absorbent material comprises nitrocellulose, polysulfones, polycarboxylic acids, or filter paper.
40. Use of an antibody specific to Arresten for the diagnosis, treatment, or prognosis of ovarian cancer in a subject.
41. Methods comprising any features, combinations of features, and/or sub-combinations of features described herein.
42. Kits comprising any features, combinations of features, and/or sub-combinations of features described herein.
43. Devices comprising any features, combinations of features, and/or sub-combinations of features described herein.
44. Uses of any features, combinations of features, and/or sub-combinations of features described herein.

### Numbered Embodiments—Example B

1. A method for diagnosing ovarian cancer in a human female subject, comprising:

isolating Arresten that is present in a diagnostic sample of the subject by immobilizing the Arresten on a solid surface;

forming a complex of the immobilized Arresten with a primary antibody specific for Arresten, said complex coupled with an enzyme to form an enzyme complex;

incubating the enzyme complex with a substrate for the enzyme;

measuring a detectable signal produced by the enzyme acting on the substrate; and

calculating a level of Arresten in the diagnostic sample based on the measurement of the detectable signal,

wherein the level of Arresten elevated above a predetermined cut-off value is diagnostic of ovarian cancer in the subject.

2. The method of embodiment 1, wherein the enzyme in the enzyme complex is conjugated to at least one of the following:

(a) the primary antibody;

(b) a secondary antibody that binds to the primary antibody; and

(c) a protein that binds to a biotin labelling: (i) the primary antibody or (ii) the secondary antibody.

3. The method of embodiment 2, wherein the protein is streptavidin or avidin, and wherein the streptavidin or avidin is native or modified by glycosylation or deglycosylation.
4. The method of any one of embodiments 1-3, wherein the immobilized Arresten is directly immobilized to the solid surface or indirectly immobilized to the solid surface through a capture antibody that is coated on the solid surface and specifically binds to Arresten.
5. The method of any one of embodiments 1-4, further comprising comparing the detectable signal to a calibration data set generated using a calibrator at multiple concentrations or levels to assess amount, concentration, or level of Arresten in the diagnostic sample, wherein the calibrator is recombinant human Arresten.
6. The method of any one of embodiments 1-5, wherein the detectable signal is measured by colorimetry or by a method selected from the group consisting of immunofluorescence, bioluminescence, and chemiluminescence.
7. The method of any one of embodiments 1-6, wherein:

the predetermined cut-off value is 80-100 pg/ml; or

the predetermined cut-off value is set so that the method has: (a) a sensitivity of at least about 60%; (b) a specificity of at least about 75%; or (c) a sensitivity of at least about 60% and a specificity of at least about 75%.

8. The method of any one of embodiments 1-7, wherein the predetermined cut-off value is set so that the method has a specificity or sensitivity of about 90% or greater.
9. The method of embodiment 7, wherein the predetermined cut-off value is determined based on a receiver operating characteristic (ROC) curve with an area under the curve (AUC) of at least about 0.70.
10. The method of embodiment 9, wherein the AUC is at least about 0.85.
11. The method of embodiment 9, wherein the predetermined cut-off value is based on at least one of the following: (a) a type of ovarian cancer and (b) a stage of ovarian cancer.
12. The method of embodiment 11, wherein the predetermined cut-off value is specific to stage II ovarian cancer and set so that the method has a sensitivity of least about 60%.
13. The method of embodiment 12, wherein the subject is asymptomatic of ovarian cancer.
14. The method of any one of embodiments 1-13, wherein the diagnosing is to screen the subject for ovarian cancer, who is asymptomatic of ovarian cancer.
15. A method for treating ovarian cancer in a subject, comprising:

administering to the subject a treatment comprising at least one of surgical debulking, chemotherapy, and radiation therapy, wherein the subject was diagnosed with ovarian cancer by a process comprising:

(a) obtaining a serum or plasma sample from the subject;

(b) analyzing the sample for Arresten expression; and

(c) detecting Arresten expression elevated above normal, thereby diagnosing the subject with ovarian cancer.

16. The method of embodiment 15, wherein the process has a sensitivity of at least about 60% and a specificity of at least about 75%.
17. The method of embodiment 16, wherein the process is based on a receiver operating characteristic (ROC) curve with an area under the curve (AUC) of at least about 0.70.
18. A diagnostic kit for assessing risk of ovarian cancer in a female subject by measuring a level of Arresten expression in a biological sample obtained from the subject, the kit comprising:

(a) a capture antibody that is capable of specifically binding to human Arresten, thereby isolating human Arresten from the biological sample;

(b) a solid matrix to which the capture antibody will bind;

(c) a detection antibody that is capable of specifically binding to human Arresten and has a label for generating a detectable signal;

(d) a recombinant human Arresten standard for calibration; and

(e) at least one reagent suitable for generating the detectable signal in cooperation with the label, thereby detecting a level of human Arresten in the biological sample.

19. The diagnostic kit of embodiment 18, further comprising:

(a) a second capture antibody that is capable of specifically binding to human CA125, thereby isolating human CA125 from the biological sample;

(b) a second detection antibody that is capable of specifically binding to human CA125, and has a second label for generating a second detectable signal;

(c) a recombinant human CA125 standard for calibration; and

(d) at least one second reagent suitable for generating the second detectable signal in cooperation with the second label, thereby detecting expression of human CA125.

20. The diagnostic kit of embodiment 19, wherein:

a predetermined cut-off value of the human Arresten is 80-100 pg/ml, and

a predetermined cut-off value for the human CA125 is 35-100 U/ml.

21. The diagnostic kit of any one of embodiments 18-20, which is adapted for point-of-care testing.
22. The method of any one of embodiments 1-14, further comprising the step of obtaining the diagnostic sample from the subject.
23. The method of any one of embodiments 1-17, wherein the ovarian cancer is an epithelial carcinoma.
24. The method of embodiment 23, wherein the epithelial carcinoma is a serous, mucinous, endometrioid, clear cell, transitional cell, squamous cell, or mixed epithelial neoplasm.
25. The method of any one of embodiments 1-17, wherein the ovarian cancer is primary ovarian cancer.
26. The method of any one of embodiments 1-17, wherein the ovarian cancer is high-grade serous ovarian cancer.
27. The method of any one of embodiments 1-14, wherein the diagnostic sample is a serum, plasma, or urine sample.
28. The method of any one of embodiments 1-14, comprising use of a paper-based ELISA.
29. The method of any one of embodiments 1-14, wherein the predetermined cut-off value is 1.2-2 times greater than normal level.
30. The method of any one of embodiments 1-17, wherein the subject is at least one of the following:

(a) pre-menopausal;

(b) asymptomatic of ovarian cancer;

(c) not carrying a mutation of BRCA1 or BRCA2 gene;

(d) suffering from a non-malignant gynecologic disease, a peritoneal, pleural, or musculoskeletal inflammatory disorder, a pelvic inflammatory disease, a liver, renal, or cardiac disease, or an advanced adenocarcinoma.

31. The method of any one of embodiments 1-17, wherein the ovarian cancer is a type II ovarian cancer.
32. The method of embodiment 31, wherein the type II ovarian cancer is at stage II, Ma, or IIIb.
33. The method of any one of embodiments 1-17, wherein the ovarian cancer is undetectable by a CA125 test or a transvaginal ultrasound test.
34. The method of any one of embodiments 1-14, further comprising assaying the diagnostic sample of the subject for at least one additional biomarker selected from CA125 and HE4.
35. The method of embodiment 34, wherein the at least one additional biomarker is CA125 with a predetermined cut-off value of 35-100 U/ml.
36. An in vitro method for using Arresten as a biomarker for screening a human female subject for ovarian cancer, comprising:

exposing a diagnostic sample of the subject to a surface that is coated with a first anti-Arresten antibody;

adding a second anti-Arresten antibody, wherein the first and second antibodies bind to different epitopes of Arresten;

measuring an amount of the second antibody bound to Arresten isolated from the diagnostic sample of the subject; and

determining a level of Arresten in the diagnostic sample based on the amount of the second antibody,

wherein the level of Arresten elevated above a predetermined cut-off value is indicative of ovarian cancer in the subject.

37. A method of preparing an Arresten-antibody complex for diagnosing and treating ovarian cancer in a human female subject, comprising:

obtaining a serum or plasma sample from the subject;

providing a capture antibody and a detection antibody that specifically bind to Arresten, wherein the capture and detection antibodies bind to different epitopes of Arresten;

coating a support surface with the capture antibody and incubating at room temperature;

adding the sample to the support surface coated with the capture antibody, thereby contacting the capture antibody with Arresten present in the sample, and incubating at room temperature;

adding the detection antibody to Arresten bound to the capture antibody and incubating at room temperature; and

producing a complex of Arresten with the capture and detection antibodies on the support surface for detection of an elevated level of Arresten expression in the subject.

38. An Arresten-antibody complex for diagnosing and treating ovarian cancer in a human female subject, prepared in accordance with the method defined in embodiment 37.
39. A method for administering an ovarian-cancer therapy to a subject, comprising:

(a) directing a clinical laboratory to
(i) process a serum or plasma sample of the subject with a reagent containing an antibody that binds to Arresten;
(ii) measure a level of Arresten expression in the sample; and
(iii) communicate the measured level of Arresten; and

(b) administering an ovarian-cancer therapy comprising at least one of surgical debulking, chemotherapy, and radiation therapy when the measured level is elevated as compared with a reference sample.

40. A method for diagnosing and treating ovarian cancer in a subject, comprising:

obtaining a serum or plasma sample from the subject;

detecting Arresten expression in the sample elevated above normal, thereby diagnosing the subject with ovarian cancer, wherein the elevated Arresten expression is detected using an immunoassay including at least one reagent that is an anti-Arresten antibody capable of forming an antigen-antibody complex with Arresten present in the sample, and

administering to the subject a treatment comprising at least one of surgical debulking, chemotherapy, and radiation therapy.

41. A method of screening a general population for ovarian cancer, comprising: testing a plurality of asymptomatic subjects in accordance with the method of embodiment 1.

### REFERENCES

1. Siegel, R. et al., Cancer statistics. CA Cancer J Clin, 2012. 62:10-29.
2. Canadian Cancer Statistics. Canadian Cancer Society, 2016.
3. Scully, R. E. et al., Histological typing of ovarian tumours. Vol. 9. New York: Springer Berlin; 1999.
4. Torre, L. A. et al., Global cancer statistics, 2012. CA Cancer J Clin, 2015. 65:87-108.
5. Visintin, I. et al., Diagnostic markers for early detection of ovarian cancer. Clin Cancer Res, 2008. 14:1065-1072.
6. Beesley, V. L. et al., Quality of life and treatment response among women with platinum-resistant versus platinum-sensitive ovarian cancer treated for progression: a prospective analysis. Gynecol Oncol, 2014. 132:130-136.
7. Piver, M. S. et al., Five-year survival for stage IC or stage I grade 3 epithelial ovarian cancer treated with cisplatin-based chemotherapy. Gynecol Oncol, 1992. 46:357-360.
8. Jemal, A. et al., Cancer statistics. CA Cancer J Clin. 2010. 60:277-300.
9. Berkenblit, A. et al., Advances in the management of epithelial ovarian cancer. J Reprod Med, 2005. 50:426-438.
10. Soletormos, G. et al., Clinical use of cancer biomarkers in epithelial ovarian cancer-updated guidelines from the European Group on Tumour Markers. Int J Gynecol Canc, 2016. 26(1):43-51.
11. Sturgeon, C. M. et al., National Academy of Clinical Biochemistry laboratory medicine practice guidelines for use of tumour markers in testicular, prostate, colorectal, breast, and ovarian cancers. Clin Chem, 2008. 54:e11-e79.
12. Van Altena, A. M. et al., CA125 nadir concentration is an independent predictor of tumour recurrence in patients with ovarian cancer: a population-based study. Gynecol Oncol, 2010. 119:265-269.
13. Zurawski Je, V. R. et al., An initial analysis of preoperative serum CA125 levels in patients with early stage ovarian carcinoma. Gynecol Oncol. 1988. 30:7-14.
14. Duffy, M. J. et al., CA125 in ovarian cancer: European Group on Tumour Markers. Int J Gynecol Canc, 2005. 15:679-691.
15. Liu, J. et al., Anti-angiogenic agents in ovarian cancer: dawn of a new era? Curr Oncol Rep, 2011. 13:450-458.
16. Schummer, M. et al., Comparative hybridization of an array of 21500 ovarian cDNAs for the discovery of genes overexpressed in ovarian carcinomas. Gene, 1999. 238:375-385.
17. Moor, R. G. et al., Serum HE4 levels are less frequently elevated than CA125 in women with benign gynecologic disorders. Am J Obstet Gynecol, 2014. 206(4):351.e1-351.e8.
18. Li, J., et al., HE4 as a biomarker for ovarian and endometrial cancer management. Expert Rev Mol Dia. 2009. 9(6):555-566.
19. Moor, R. G. et al., The use of multiple novel tumour biomarkers for the detection of ovarian carcinoma in patients with pelvic mass. Gynecol Oncol, 2008. 108(2):402-408.
20. Molina, R. et al., HE4 a novel tumour marker for ovarian cancer: comparison with CA 125 and ROMA algorithm in patients with gynaecological diseases. Tumour Biol, 2011. 32:1087-1095.
21. Zheng, H. et al., Serum HE4 as a useful biomarker in discriminating ovarian cancer from benign pelvic disease. Int J Gynecol Cancer, 2012. 22:1000-1005.
22. Drapkin, R. et al., Human epididymis protein 4 (HE4) is a secreted glycoprotein that is overexpressed by serous and endometrioid ovarian carcinomas. Cancer Res, 2005. 65(6):2162-2169.
23. Simmons, A. R. et al., The emerging role of HE4 in the evaluation of epithelial ovarian and endometrial carcinomas. Oncology (Williston Park), 2013. 27:548-556.
24. Moor, R. G. et al., A novel multiple biomarker assay utilizing HE4 and CA125 for the prediction of ovarian cancer in patients with pelvic mass. Gynecol Oncol, 2009. 112:40-46.

Van Gorp, T. et al., HE4 and CA125 as a diagnostic test in ovarian cancer: prospective validation of the Risk of Ovarian malignancy Algorithm. Br J Cancer, 2011. 104:863-870.
26. Dayyani, F. et al., Diagnostic performance of risk of ovarian malignancy algorithm against CA125 and HE4 in connection with ovarian cancer. Int J Gynecol Canc, 2016.
27. Wang, J. et al., Diagnostic accuracy of serum HE4, CA125 and ROMA in patients with ovarian cancer: a meta-analysis. Tumour Biology, 2014. 35(6):6127-6138.
28. Montagnana, M. et al., The ROMA (Risk of Ovarian malignancy Algorithm) for estimating the risk of epithelial ovarian cancer in women presenting with pelvic mass: is it really useful? Clin Chem Lab Med, 2011. 49:521-525.
29. Karlsen, M. A. et al., Evaluation of HE4, CA125, risk of ovarian malignancy algorithm (ROMA) and risk of malignancy index (RMI) as diagnostic tools of epithelial ovarian cancer in patients with pelvic mass. Gynecol Oncol, 2012. 127:379-383.
30. Ueland, F. R. et al., A perspective on ovarian cancer biomarkers: past, present and yet-to-come, Diagnostics, 2017. 7-14.
31. Soussi, T. & Wiman, K. G., Shaping genetic alterations in human cancer: the p53 mutation paradigm. Cancer Cell, 2007. 12:303-312.
32. Kandoth, C. et al., Mutational landscape and significance across 12 major cancer types. Nature, 2013. 502:333-339.
33. Bieging, K. T. et al., Unravelling mechanisms of p53-mediated tumour suppression. Nat Rev Cancer, 2014. 14:359-370.
34. Canadian Cancer Statistics. Canadian Cancer Society, 2020.
35. Oren, M. & Rotter, V., Mutant p53 gain-of-function in cancer. Cold Spring Harb Perspect Biol, 2010. 2:a001107.
36. Leroy, B. et al., The TP53 website: an integrative resource centre for the TP53 mutation database and TP53 mutant analysis. Nucleic Acids Res, 2013. 41:D962-969.
37. Hahn, W. C. et al., Modelling the molecular circuitry of cancer. Nat Rev Cancer, 2002. 2:331-341.
38. Sherr, C. J. et al., The RB and p53 pathways in cancer. Cancer Cell, 2002. 2:103-112.
39. Kusume, T. et al., The p16-cyclin D1/CDK4-pRb pathway and clinical outcome in epithelial ovarian cancer. Clin Cancer Res. 1999. 5:4152-4157.
40. Fujita, M. et al., Alteration of p16 and p15 genes in common epithelial ovarian tumours. Int J Cancer. 1997. 74:148-155.
Katsaros, D. et al., Methylation of tumour suppressor gene p16 and prognosis of epithelial ovarian cancer. Gynecol Oncol. 2004. 94:685-692.
42. Colorado, P. C. et al., Anti-angiogenic cues from vascular basement membrane collagen, Cancer Res, 2000. 60:2520-2526.
43. Nyberg, P. et al., Characterization of the anti-angiogenic properties of arresten, an α1β1 integrin-dependent collagen-derived tumour suppressor. Exp Cell Res, 2008. 314:3292-3305.
44. Teodoro, J. G., et al., p53-mediated inhibition of angiogenesis through up-regulation of a collagen prolyl hydroxylase. Science, 2006. 313(5789):968-71.
45. Assadian, S., et al., p53 inhibits angiogenesis by inducing the production of Arresten. Cancer Res., 2012. 72(5):1270-9.
46. Bell, D. et al., Integrated genomic analyses of ovarian carcinoma. Nature, 2011. 474:609-615.
47. Patch, A. M. et al., Whole-genome characterization of chemoresistant ovarian cancer. Nature, 2015. 521:489-494.
48. Rosen, B. et al., The impacts of neoadjuvant chemotherapy and of debulking surgery on survival from advanced ovarian cancer. Gynecol Oncol, 2014. 134(3): 462-7.
49. Chi, D. S. et al., An analysis of patients with bulky advanced stage ovarian, tubal, and peritoneal carcinoma treated with primary debulking surgery (PDS) during an identical time period as the randomized EORTC-NCIC trial of PDS vs neoadjuvant chemotherapy (NACT). Gynecol Oncol, 2012. 124(1): 10-4.
50. Bast, R. C., Jr. et al., A radioimmunoassay using a monoclonal antibody to monitor the course of epithelial ovarian cancer. N Engl J Med, 1983. 309(15):883-7.
51. Van Calster, B. et al., A novel approach to predict the likelihood of specific ovarian tumour pathology based on serum CA-125: a multicenter observational study. Cancer Epidemiol Biomarkers Prev, 2011. 20(11):2420-8.
52. Bonfrer, J. M. et al., Clinical evaluation of the Byk LIA-mat CA125 II assay: discussion of a reference value. Clin Chem, 1997. 43(3):491-7.
53. Kohler, G. and Milstein, C., European Journal of Immunology, 1976. 6:511-519.
54. Clackson, T. et al. Nature, 1991. 352(6336):624-628.
55. Marks, J. D. et al., J. Mol. Biol., 1991. 222(3):581-597.
56. Siegel, R. et al., (2012). Cancer treatment and survivorship statistics. CA Cancer J Clin, 2012. 62(4):220-241.

All publications mentioned herein are hereby incorporated by reference in their entireties. While the foregoing invention has been described in some detail for purposes of clarity and understanding, it will be appreciated by one skilled in the art, from a reading of the disclosure, that various changes in form and detail can be made without departing from the true scope of the invention in the appended claims.

What is claimed is:

1. A method for diagnosing ovarian cancer in a human female subject, comprising:
isolating Arresten that is present in a diagnostic sample of the subject by immobilizing the Arresten on a solid surface;
forming a complex of the immobilized Arresten with a primary antibody specific for Arresten, said complex coupled with an enzyme to form an enzyme complex;
incubating the enzyme complex with a substrate for the enzyme;
measuring a detectable signal produced by the enzyme acting on the substrate; and
calculating a level of Arresten in the diagnostic sample based on the measurement of the detectable signal,
wherein the level of Arresten elevated above a predetermined cut-off value is diagnostic of ovarian cancer in the subject.

2. The method of claim 1, wherein the enzyme in the enzyme complex is conjugated to at least one of the following:
(a) the primary antibody;
(b) a secondary antibody that binds to the primary antibody; and
(c) a protein that binds to a biotin labelling: (i) the primary antibody or (ii) the secondary antibody.

3. The method of claim 2, wherein the protein is streptavidin or avidin, and wherein the streptavidin or avidin is native or modified by glycosylation or deglycosylation.

4. The method of claim 1, wherein the immobilized Arresten is directly immobilized to the solid surface or indirectly immobilized to the solid surface through a capture antibody that is coated on the solid surface and specifically binds to Arresten.

5. The method of claim 1, further comprising comparing the detectable signal to a calibration data set generated using a calibrator at multiple concentrations or levels to assess amount, concentration, or level of Arresten in the diagnostic sample, wherein the calibrator is recombinant human Arresten.

6. The method of claim 5, wherein the detectable signal is measured by colorimetry or by a method selected from the group consisting of immunofluorescence, bioluminescence, and chemiluminescence.

7. The method of claim 1, wherein:
the predetermined cut-off value is 80-100 pg/ml; or
the predetermined cut-off value is set so that the method has: (a) a sensitivity of at least about 60%; (b) a specificity of at least about 75%; or (c) a sensitivity of at least about 60% and a specificity of at least about 75%.

8. The method of claim 1, wherein the predetermined cut-off value is set so that the method has a specificity or sensitivity of about 90% or greater.

9. The method of claim 7, wherein the predetermined cut-off value is determined based on a receiver operating characteristic (ROC) curve with an area under the curve (AUC) of at least about 0.70.

10. The method of claim 9, wherein the AUC is at least about 0.85.

11. The method of claim 9, wherein the predetermined cut-off value is based on at least one of the following: (a) a type of ovarian cancer and (b) a stage of ovarian cancer.

12. The method of claim 11, wherein the predetermined cut-off value is specific to stage II ovarian cancer and set so that the method has a sensitivity of least about 60%.

13. The method of claim 12, wherein the subject is asymptomatic of ovarian cancer.

14. The method of claim 1, wherein the diagnosing is to screen the subject for ovarian cancer, who is asymptomatic of ovarian cancer.

15. A method for treating ovarian cancer in a subject, comprising:
administering to the subject a treatment comprising at least one of surgical debulking, chemotherapy, and radiation therapy, wherein the subject was diagnosed with ovarian cancer by a process comprising:
(a) obtaining a serum or plasma sample from the subject;
(b) analyzing the sample for Arresten expression; and
(c) detecting Arresten expression elevated above normal, thereby diagnosing the subject with ovarian cancer.

16. The method of claim 15, wherein the process has a sensitivity of at least about 60% and a specificity of at least about 75%.

17. The method of claim 16, wherein the process is based on a receiver operating characteristic (ROC) curve with an area under the curve (AUC) of at least about 0.70.

18. A diagnostic kit for assessing risk of ovarian cancer in a female subject by measuring a level of Arresten expression in a biological sample obtained from the subject, the kit comprising:
(a) a capture antibody that is capable of specifically binding to human Arresten, thereby isolating human Arresten from the biological sample;
(b) a solid matrix to which the capture antibody will bind;
(c) a detection antibody that is capable of specifically binding to human Arresten and has a label for generating a detectable signal;
(d) a recombinant human Arresten standard for calibration; and
(e) at least one reagent suitable for generating the detectable signal in cooperation with the label, thereby detecting a level of human Arresten in the biological sample.

19. The diagnostic kit of claim 18, further comprising:
(a) a second capture antibody that is capable of specifically binding to human CA125, thereby isolating human CA125 from the biological sample;
(b) a second detection antibody that is capable of specifically binding to human CA125, and has a second label for generating a second detectable signal;
(c) a recombinant human CA125 standard for calibration; and
(d) at least one second reagent suitable for generating the second detectable signal in cooperation with the second label, thereby detecting expression of human CA125.

20. The diagnostic kit of claim 19, wherein:
a predetermined cut-off value of the human Arresten is 80-100 pg/ml, and
a predetermined cut-off value for the human CA125 is 35-100 U/ml.
